(19) United States
(12) Patent Application Publication
(10) Pub. No.: US 2010/0286747 A1
(43) Pub. Date: Nov. 11, 2010

(54) METHODS FOR APPLYING BRAIN SYNCHRONIZATION TO EPILEPSY AND OTHER DYNAMICAL DISORDERS

(75) Inventors: Shivkumar Sabesan, Tempe, AZ (US); Leonidas D. Iasemidis, Scottsdale, AZ (US)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: Arizona Board of Regents, Acting for and on Behalf of Arizona State University, Tempe, AZ (US)

(21) Appl. No.: 12/680,509

(22) PCT Filed: Sep. 29, 2008
(86) PCT No.: PCT/US08/78178

§ 371 Date: Jul. 28, 2010

Related U.S. Application Data
(60) Provisional application No. 60/995,884, filed on Sep. 28, 2007.

(51) Int. Cl. A61N 1/00; G06F 19/00; G06F 17/18

(52) U.S. Cl. 607/45; 702/19; 702/179

(57) ABSTRACT

For analyzing a multi-component system, a method acquires a plurality of signals, each having a different spatial location of the multi-component system, and generates dynamic profiles for each of the plurality of signals. Each of the plurality of dynamic profiles reflects dynamic characteristics of the corresponding signal in accordance with each one of a plurality of dynamic measures. The method selects pairs of dynamic profiles from the acquired dynamic profiles based on a predetermined level of synchronization and generates a statistical measure for each of the selected plurality of pairs of dynamic profiles. The method characterizes state dynamics of the multi-component system as a function of at least one of the generated statistical measures, and generates a signal indicative of the characterized state dynamics of the multi-component system. The method enables seizure detection, seizure prediction, seizure focus localization, differential diagnosis of epilepsy and evaluation of seizure intervention strategies

### CROSS-REFERENCE TO RELATED APPLICATIONS

This Application claims the benefit of the filing date and priority to the following patent application, which is incorporated herein by reference to the extent permitted by law:

U.S. Provisional Application Ser. No. 60/995,884, entitled “APPLICATION OF BRAIN SYNCHRONIZATION TO EPILEPSY AND OTHER DYNAMICAL DISORDERS”, filed on Sep. 28, 2007.

### FIELD OF THE INVENTION

The present invention generally relates to the field of signal processing and, more particularly, to methods for applying brain synchronization to epilepsy and other dynamic disorders.

### BACKGROUND OF THE INVENTION

Epileptic seizures are manifestations of epilepsy, a neurological dynamical disorder second only to stroke. About one third (⅓) of the 50 million people with epilepsy have seizures that are not controlled by anti-convulsant medication. One of the most disabling aspects of epilepsy is the seemingly unpredictable nature of seizures. If seizures cannot be controlled, a patient experiences major limitations in family, social, educational, and vocational activities. These limitations have profound effects on the patient's quality of life, as well as on his or her family. In addition, status epilepticus, a life-threatening condition where seizures occur continuously, is treated only upon extreme intervention.

A general belief in the medical community has been that epileptic seizures could not be anticipated. Seizures were assumed to be abrupt transitions that occurred randomly over time. However, theories based on reports from clinical practice and scientific intuition pointed to the direction of seizure predictability. Various feelings of auras, such as patients' reports of sensations of an upcoming seizure, have been documented in the medical literature. Changes in the cerebral blood flow were noted to occur prior to seizures. Deterministically predictable occurrences of seizures (reflex seizures) in a small minority (about 3 to 5%) of epileptic patients have been reported as a result of various sensory stimuli. These theories and facts have implied that seizures might be predictable.

The ability to predict epileptic seizures well prior to their occurrences may lead to novel diagnostic tools and treatment of epilepsy. Evaluation of anti-epileptic drugs and protocols, in terms of duration of patients' seizure susceptibility periods and/or preictal (before a seizure) periods detected by seizure prediction algorithms, may lead to the design of new, more effective and with less side effects drugs for early disruption of the epileptic brain's route towards a seizure. Electromagnetic stimulation and/or administration of anti-epileptic drugs (AEDs) at the beginning of the preictal period may disrupt the observed dynamical entrainment of normal brain sites with the epileptogenic focus (the area that first exhibits the electrographic onset of ictal activity), and lead to a significant reduction of epileptic seizures. Aside from their immediate clinical applications to epilepsy, successful seizure prediction and control algorithms could be useful for investigations into a wide variety of other complex, nonstationary and spatio-temporal biological and physical systems that undergo intermittent transitions.

New signal processing methodologies, based on the mathematical theory of nonlinear dynamics, were discovered in the 1980s for the study of spontaneous formation of organized spatial, temporal or spatiotemporal patterns in physical, chemical and biological systems. These methodologies typically quantify the signal structure from the perspective of dynamical invariants and are a drastic departure from the signal processing techniques based on the linear model (e.g. Fourier analysis).

In 1988, a research group at the University of Michigan, Ann Arbor, led by Leon D. Iasemidis, J. Chris Sackellares and W. J. Williams, reported the first application of nonlinear dynamics to clinical epilepsy. This University of Michigan group analyzed continuous, multichannel, preictal (before seizure), ictal (during seizure) and postictal (after seizure) electroencephalogram (EEG) from epileptic patients with temporal lobe epilepsy devising new and modifying existing brain-related measures from the theory of chaos to quantify the rate of divergence of trajectories of these measures for the analysis of EEG in epilepsy. A central concept was that seizures represented transitions of the epileptic brain from its “normal” less ordered (chaotic) state to an abnormal, more ordered state, and back to a “normal” state along the lines of chaos-to-order-to-chaos transitions.

The dynamical modeling hypothesis changed some long-held beliefs about seizures. Iasemidis et al. reported the first evidence that the transition to epileptic seizures may be consistent with a deterministic process, and that an ictal EEG can be better modeled as an output of a nonlinear than that of a linear system. The existence of long-term preictal periods (order of minutes) was demonstrated using nonlinear dynamical analysis of subdural arrays, and raised the feasibility of seizure prediction algorithms by monitoring relevant characteristics of the brain-related measures, such that a temporal evolution of the short-term Lyapunov exponents (STLₘₐₓ). The possibility of focus localization and seizure detection was also reported with the same technique in the 1990s. Since these initial results, other research groups started to work in the area of seizure prediction. One research group investigated the spatio-temporal dynamics of the epileptic focus in 1994. Another research group directed its attention to the time structure of inter-ictal spikes. Still another research group, who had been developing neurophysiology-driven dynamical models for EEG activity since the late 70s, quantified state bifurcations of these model dynamics in epileptogenesis.

Iasemidis et al. improved their STLₘₐₓ technique with the use of optimization techniques and a critical mass hypothesis to predict seizures. A fundamental issue that surfaced through this group's investigations in seizure predictions was the importance to locate and use only the measures or channels that carry information about an impending seizure. Changes in the spatio-temporal structure of the EEG can in principle also be quantified by sophisticated linear methods involving wavelet decompositions, coherence, and pattern recognition methods such as artificial neural networks, and fuzzy approaches. Several research groups have employed this approach towards the detection of the preictal period. However, no prospective results on seizure prediction (i.e. seizure prediction from long-term continuous EEG data using information only from past seizure occurrences) have been reported in the literature. An important conclusion from all these different techniques is an accumulation of evidence that there are measurable differences in the EEG prior to seizure onset that can be utilized for epileptic seizure prediction.

Iasemidis et al. reported a progressive preictal increase of spatiotemporal entrainment/synchronization between critical sites of the brain as the precursor of epileptic seizures. The algorithm used was based on the convergence of short-term maximum Lyapunov exponents (STLₘₐₓ) among critical electrode sites selected adaptively. This observation has been successfully implemented in the prospective prediction of epileptic seizures. Global optimization techniques were applied for selecting the critical groups of electrode sites to observe preictal entrainment. Seizure anticipation times of about 71.7 minutes with a false prediction rate of 0.12 per hour were reported. To further relate these findings to the mechanism of epileptogenesis, Iasemidis et al. found that majority of seizures in patients with temporal lobe epilepsy (TLE) irreversibly reset (disentrain) postictally the observed preictal dynamical entrainment. This supports the hypothesis that seizures do not occur as long as there is no need to reset the brain. Moreover, Iasemidis et al. have also shown through simulations that, in chaos-to-order-to-chaos transitions of general models of spatially coupled chaotic oscillators, with an increase/decrease of coupling convergence/divergence of the STLₘₐₓ resembles the observed preictal entrainment and postictal dynamical disentrainment of the STLₘₐₓ of critical brain sites. In addition, the models exhibited hysteresis, a phenomenon that is also observed in the epileptic transition into and out of seizures. This dynamical view leads to a characterization of the seizure itself as a mechanism that the brain has developed to reset the preictal entrainment when a critical mass of sites, or a mass of specific, critical sites, is recruited.

Therefore, three central results about epileptic seizures have emerged. First, seizures have been shown to be manifestations of recruitment of brain sites in an abnormal hyper-synchronization. The onset of such recruitment occurs long before a seizure and progressively may culminate into a seizure. Seizures appear to be bifurcations of a neural network that involve a progressive coupling of the focus with the normal brain sites during a preictal period that may last days to tens of minutes. Thus, identification of such a preictal period may constitute the basis for predicting an impending seizure well in advance as well as may lead to accurate localization of the epileptogenic focus. Second, postictally, time-irreversible resetting of the observed preictal dynamical recruitment occurs (via a hysteretic loop). Preictal and postictal periods can be mathematically defined and detected from the EEG. Complete or partial resetting of the preictal entrainment of the epileptic brain after the seizure may affect the route to a subsequent seizure. This may contribute to the observed nonstationary nature of the seizure occurrences. Therefore, it is expected that estimation of the magnitude of resetting at the seizure may improve our understanding of the brain's route to subsequent seizures, and may even lead to better seizure prediction and control. Third, through control-oriented modeling, a feedback control view of epileptic seizures has been postulated, wherein epileptic seizures are hypothesized to be a result of the inability of the internal feedback/regulatory mechanisms of the brain to track excessive synchronization changes between the epileptogenic focus and other brain areas prior to a seizure.

Therefore, there is a need for a method for applying brain entrainment/synchronization to epilepsy and other dynamic disorders, so as to improve the detections, predictions and localizations of seizures.

### SUMMARY OF THE INVENTION

A technical advance is achieved by one or more embodiments, methods implemented according to teaching of the present invention. For purposes of illustration and not limitation the embodiments are described below in terms of methods that detect and predict epileptic seizures, localize the epileptogenic focus/zone in the epileptic brain, measure the efficacy of electrical/magnetic/drug-based control of epileptic seizures and determine the need for subsequent intervention to efficiently control seizures including the highly pathological condition of Status Epilepticus, and serve as a reliable diagnostic tool for epilepsy versus other brain disorders such as but not limited to metabolic encephalopathy, and pseudo-seizures.

In one embodiment, a method for analyzing a multi-component system is provided. The method acquires a plurality of signals, each signal associated with a different spatial location of a portion of the multi-component system, and generates a plurality of dynamic profiles for each of the plurality of signals. Each of the plurality of dynamic profiles reflects dynamic characteristics of the corresponding signal in accordance with each one of a plurality of dynamic measures. The method selects a plurality of pairs of dynamic profiles from the acquired plurality of dynamic profiles based on a predetermined level of synchronization and generates a statistical measure for each of the selected plurality of pairs of dynamic profiles. The method characterizes state dynamics of the multi-component system as a function of at least one of the generated statistical measures, and generates a signal indicative of the characterized state dynamics of the multi-component system.

In another embodiment, the indicative signal generated by the above described method is used to at least one of monitor dynamic transitions of the multi-component system, identify spatial locations of interest in the multi-component system, differentiate the state dynamics of the multi-component system from other dynamics of similar systems, evaluate and determine a treatment efficacy for the multi-component system, and identify a susceptibility of the multi-component system to a predetermined condition.

In another embodiment, the multi-component system is an epileptic brain and the signal is a seizure warning when the generated statistical measures exceed at least one of the predetermined threshold values. Moreover, the order of synchronization of the plurality of generated statistical measures is determined to characterize the state dynamics of the multi-component system.

In another embodiment, a computer-readable medium contains a program adapted to cause a data processing system to execute the above-noted method. In this regard, the computer-readable medium may be a computer-readable medium, such as solid-state memory, magnetic memory such as a magnetic disk, optical memory such as an optical disk, or a computer-readable transmission medium, such as a modulated wave (such as radio frequency, audio frequency or optical frequency modulated waves) or a modulated downloadable bit stream that can be received by a computer via a network or a via a wireless connection.

Other methods, features, and advantages will be or will become apparent to one with skill in the art upon examination of the following figures and detailed description. It is intended that all such additional systems, methods, features, and advantages be included within this description, be within the scope of the present disclosure, and be protected by the accompanying claims.

### BRIEF DESCRIPTION OF THE DRAWINGS

The accompanying drawings, which are incorporated in and constitute part of the present specification, illustrate exemplary embodiments. The present disclosure may be better understood by reference to one or more of these drawings in combination with the detailed description of specific embodiments presented herein. In the drawings:

FIG. 1 is a schematic diagram illustrating an exemplary embodiment of depth and subdural electrode placements in a brain;

FIG. 2 is a set of graphs illustrating T-index profiles generated from STLmax profiles of dynamically entrained pairs of electrode sites prior to different seizures in Patient 1 and Patient 2;

FIG. 3 is a graph illustrating dynamical synchronization of entrained pairs of electrode sites prior to a seizure in Patient 1 estimated from the measures of STLmax, Phase and energy;

FIG. 4 is a graph illustrating an estimation of the seizure predictability time Tₚ from the T-index profile of selected critical electrode sites for the seizure of FIG. 3;

FIG. 5 is a flow diagram illustrating an embodiment of an automated seizure prediction method;

FIG. 6 is a flow diagram illustrating an embodiment of another automated seizure prediction method;

FIG. 7 is a graph illustrating dynamical transitions (warnings) of Algorithm 1 in Patient 1;

FIG. 8 is a graph illustrating dynamical transitions (warnings) of Algorithm 1 in Patient 2;

FIG. 9 is a graph illustrating dynamical transitions (warnings) of Algorithm 2 in Patient 1;

FIG. 10 is a graph illustrating dynamical transitions (warnings) of Algorithm 2 in Patient 2;

FIG. 11 is a flow diagram illustrating an embodiment of an automated seizure detection method;

FIG. 12 is a graph illustrating seizure detection results in Patient 1;

FIG. 13 is a flow diagram illustrating an embodiment of an automated seizure focus localization method;

FIG. 14 is a graph illustrating long-term spatio-temporal dynamics per area of an amount of synchronization (S) and a rate of resetting (Sᵣᵣ) over a 10-day period from Patient 1;

FIG. 15 is a flow diagram illustrating an embodiment of a method for evaluating seizure control efficacy;

FIG. 16 is a graph illustrating warning-based stimulation of the epileptic brain of a rat leading to reduction of seizure frequency;

FIG. 17 is a set of graphs illustrating two EEG traces of metabolic encephalopathy (ME) in a female patient (Column A1) and two corresponding EEG traces of Status Epilepticus (SE) in a young patient (Column A2);

FIG. 18 is a flow diagram illustrating an embodiment of a method for differential diagnosis of status epilepticus;

FIG. 19 is a set of graphs illustrating an amount of synchronization S (t) versus time (in minutes) for an SE patient and a metabolic encephalopathy patient;

FIG. 20 is a flow diagram illustrating an embodiment of a method for estimating seizure susceptibility;

FIG. 21 is a block diagram illustrating an embodiment of a system for monitoring dynamical behavior of the multi-component system in accordance with the described exemplary embodiments; and

FIG. 22 is a block diagram illustrating an overview of the applications related to the described methods.

### DETAILED DESCRIPTION

Reference will now be made in detail to embodiments as illustrated in the accompanying drawings. Wherever possible, the same reference numbers will be used throughout the drawings and the following description to refer to the same or like parts. It should be appreciated by those of skill in the art that the techniques disclosed in the examples which follow represent techniques discovered by the inventors to function in practice, and thus can be considered to constitute embodiments. However, those of skill in the art should, in light of the present disclosure, appreciate that many changes can be made in the specific embodiments which are disclosed and still obtain a like or similar result without departing from the spirit and scope described herein.

The detection of epilepsy, which even today includes visual scanning of EEG recordings for spikes and seizures, is a very time consuming process, especially in the case of long recordings. In addition, EEG signal morphology is so highly variable across patients and brain states that any diagnosis becomes highly subjective and often, disagreement between physicians occurs on the same EEG record. Therefore, the need to objectively extract parameters from EEG that are characteristic of a seizure cannot be overemphasized. The early methods of automatic seizure detection were based on characterizing the EEG spectral estimation using Fourier Transforms or parametric methods such as autoregressive (AR) modeling. Since the EEG signals are non-stationary, the classical signal processing methods did not produce a reliable detection scheme. Several efforts using nonlinear dynamics to detect seizures were introduced. A study was conducted to assess the accuracy of recurrent neural networks (RNN) employing Lyapunov exponents in detection seizure in the EEG signals. The traditional method of logistic regression was compared to the more advanced neural network techniques, as mathematical tools for developing classifiers for the detection of epileptic seizure in multi-channel EEG. Time-frequency analysis of EEG signals is typically configured for detecting the information on alertness and drowsiness using spectral densities of discrete wavelet transform (DWT) coefficients. As compared to the conventional method of spectral analysis, wavelet analysis of EEG signals has improved the seizure detection performance. However, besides the need for massive data to train such algorithms, the reported level of performance (sensitivity and specificity-wise) of these algorithms is still not high enough for use in clinical environments.

Without limitation to measures (parameters) of dynamics of synchronization that could have been used, in the present section, three of the most frequently utilized measures of dynamical synchronization/entrainment, namely classical energy (E), phase (Φ) and short-term Lyapunov exponent (STLₘₐₓ), are compared on the basis of their ability to detect preictal changes in the brain. In the case of the complex exponential basis signal:

x(t)=Ae^{at}e^{j(ωt+φ)}

these three quantities, correspond to Ae^{at}, ωt+φ, and α respectively, where α is the real part of the pole of the Laplace transform of x(t) and is equal to STLₘₐₓ, phase Φ=ωt+φ is the imaginary part of the pole and it depends on ω, and energy E depends on A and α. Due to the current interest in the field, and the proposed measures of energy and phase as alternatives to STLₘₐₓ for seizure prediction, it is important to comparatively evaluate seizure predictability (anticipation) capabilities of each of the three measures.

### Description of EEG Data

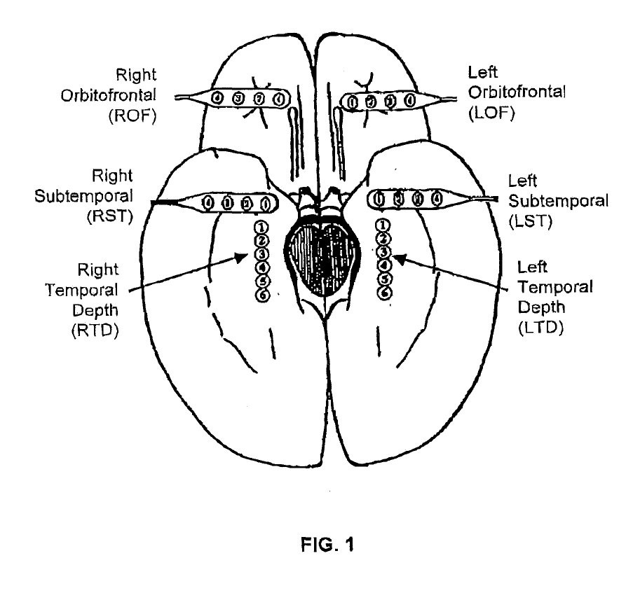

Now referring to FIG. 1, a schematic diagram illustrates depth and subdural electrode placements in a brain in accordance with the present invention. The patients in the study underwent a stereotactic placement of bilateral depth electrodes (RTD1 to RTD6 in the right hippocampus, with RTD1 adjacent to right amygdala; LTD1 to LTD6 in the left hippocampus with the LTD1 adjacent to the left amygdala; the rest of the LTD, RTD electrodes extending posterior through the hippocampi). Two subdural strip electrodes were placed bilaterally over the orbitofrontal lobes (LOFT to LOF4 in the left and ROF1 to ROF4 in the right lobe, with LOFT, ROF1 being most mesial and LOF4, ROF4 most lateral). Two subdural strip electrodes were placed bilaterally over the temporal lobes (LST1 to LST4 in the left and RST1 to RST4 in the right, with LST1, RST1 being more mesial and LST4, RST4 being more lateral).

All seizures recorded from two epileptic patients with temporal lobe epilepsy at the epilepsy monitoring unit (EMU), as shown in Table 1, were analyzed by the methodologies described below. The EEG signals were recorded from 6 different areas of the brain by 28 electrodes. Typically, periods from 3 hours before and 1 hour after each seizure were analyzed in search of dynamical synchronization and for estimation of seizure predictability times.

TABLE 1
Patient and EEG data characteristics

| | # of | Location of | | | # of | # of | Analyzed EEG |
|---|---|---|---|---|---|---|---|
| Patient | electrode | epileptogenic | Seizure | Duration of | recorded | analyzed | per seizure |
| ID | sites | focus | type | EEG (days) | seizures | seizures | (hours) |
| 1 | 28 | RTD | C | 9.06 | 24 | 24 | 4 |
| 2 | 28 | RTD | C, SC | 6.07 | 19 | 19 | 4 |

(Seizure types: C (clinical); SC (subclinical))

Video/EEG monitoring was performed using the Nicolet BMSI 4000 EEG machine. EEG signals were recorded using an average common reference with band pass filter settings of 0.1 Hz-70 Hz. The data were sampled at 200 Hz with a 10-bit quantization and recorded on VHS tapes continuously over days via 3 time-interleaved VCRs. Decoding of the data from the tapes and transfer to computer media (hard disks, DVDs, CD-ROMs and the like) was subsequently performed off-line. The seizure predictability analysis also was performed retrospectively (off-line).

Measures of EEG Synchronization

Energy Profiles (E)

The classical energy of a signal x(t) over time is calculated as the sum of its magnitude squared over a time period T, as follows:

$$E ( t ) = {\sum }_{ɛ = t}^{t + T} {\vert x ( ɛ ) \vert}^{2}$$

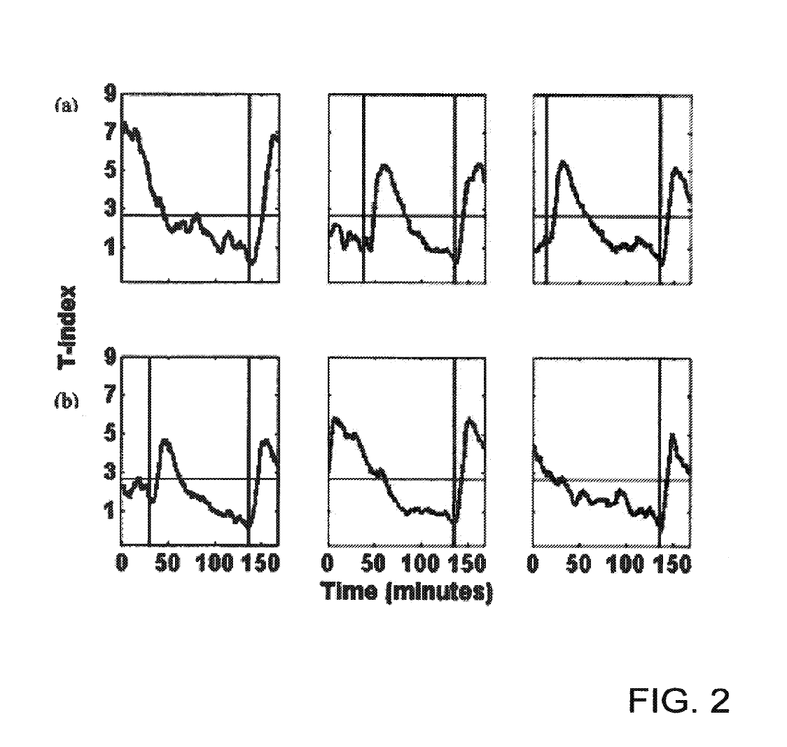

For EEG analysis, E values are calculated over consecutive non-overlapping segments of data, each segment being T seconds in duration from different locations in the brain over time t. Based on knowledge acquired from a substantial number of experiments, the duration T was selected to be equal to 10.24 seconds. Examples of E profiles over time from two electrode sites that show synchronization before a seizure are given in the left panel of FIG. 2(a). The highest E values were observed during the ictal period. This pattern roughly corresponds to the typical observation of higher amplitudes in the original EEG signal ictally.

Maximum Phase Profiles (Φₘₐₓ)

The notion of phase synchronization for two coupled frictionless harmonic oscillators oscillating at different angular frequencies ω₁ and ω₂ is known to be given by the following equation:

$$\frac{{ω}_{1}}{{ω}_{2}} = \frac{m}{n} .$$

In this classical case, phase synchronization is usually defined as locking of the phases of the two oscillators in terms of:

φ_{n,m}=nφ₁(t)−mφ₂(t)=constant,

where n and m are integers, φ₁ and φ₂ denote the phases of the oscillators, and φ_{n,m} is defined as their constant relative phase. In order to investigate synchronization in chaotic systems, this condition of phase locking is relaxed by a weaker condition of phase synchronization (since

$$\frac{{ω}_{1}}{{ω}_{2}}$$

may be an irrational real number and each system may contain power at other frequencies besides a dominant frequency):

|φ_{n,m}|=|nφ₁(t)−mφ₂(t)|<constant.

The estimation of instantaneous phases φ₁(t) and φ₂(t) is nontrivial for many nonlinear model systems, and even more difficult when dealing with noisy time series of unknown characteristics.

Different approaches have been proposed in the literature for the estimation of instantaneous phase of a signal. In the analysis that follows, an analytic signal approach for phase estimation defines the instantaneous phase of an arbitrary signal s(t) as:

$$φ ( t ) = arctan \frac{\tilde{x} ( t )}{x ( t )} , where \tilde{x} ( t ) = \frac{1}{π} P . V . {\int }_{- ∞}^{+ ∞} \frac{x ( τ )}{t - τ} d τ$$

is the Hilbert Transform of the signal x(t) (P.V. denotes the Cauchy Principal Value).

The Hilbert transformation is equivalent to a special kind of filtering of x(t) in which amplitudes of the spectral components are left unchanged, while their phases are altered by π/2, positively or negatively according to the sign of ω. For details of the practical estimation of φ(t) via the Hilbert transform, the tapering of the data by a Hamming window and subsequent utilization of the Fourier Transform. The φ(t) from EEG data are estimated per non-overlapping moving windows of 10.24 seconds in duration and per electrode site. An array of φ(t) values are returned for each window, equal in number to the number of EEG data points (x(t)) contained in the window. Then, the maximum value (Φₘₐₓ) of the phase values per window is estimated and used in subsequent analysis. An example of Φₘₐₓ profiles at two electrode sites over time is given in FIG. 2(b) (left panel). The preictal, ictal and postictal states corresponded to medium, high and lower values of Φₘₐₓ respectively. The highest Φₘₐₓ values were observed during the ictal period. Higher Φₘₐₓ values were observed during the preictal period than the postictal period. This pattern roughly corresponds to the typical observation of higher frequencies in the original EEG signal ictally, and lower EEG frequencies postictally.

### Chaos Profiles (STLₘₐₓ)

Under certain conditions, through the known method of delays, sampling of a single variable of a system over time can determine all state variables of the system that are related to an observed state of the system. In the case of the EEG, this method can be used to reconstruct a multi-component state space of the brain's electrical activity from a single EEG channel recording at a corresponding brain site. Thus, in such an embedding, each state in the state space is represented by a vector X(t), whose components are the delayed versions of the original single-channel EEG time series x(t), that is:

X(t)=(x(t), x(t+τ), . . . , x(t+(d−1)·τ))

where τ is the time delay between successive components of X(t), and d is a positive integer denoting the embedding dimension of the reconstructed state space. Plotting X(t) in the thus created state space produces the state portrait of a subsystem (brain site) of a spatially distributed system (brain) from where x(t) is recorded. A steady state of such a subsystem is considered chaotic if at least the maximum Lₘₐₓ of its Lyapunov exponents (Lₛ) is positive.

Of the many different methods typically used to estimate the embedding dimension d of an object in the state space, each has its own practical problems. The measure most often used to estimate this embedding dimension d is the state space correlation dimension v, where d≧2v+1. Methods for calculating v from experimental data are employed to approximate v in the ictal state.

The brain, being nonstationary, is never in a steady state in the strictly dynamical sense at any location. Arguably, activity at brain sites is constantly moving through approximately steady states, which are functions of certain parameter values at a given time. According to bifurcation theory, when these parameters change slowly over time, or the system is close to a bifurcation, dynamics slow down and conditions of stationarity are better satisfied. In the ictal state, temporally ordered and spatially synchronized oscillations in the EEG usually persist for a relatively long period of time (in the range of minutes). Dividing the ictal EEG into short segments ranging from 10.24 sec to 50 sec in duration, estimation of v from ictal EEG has given values between 2 and 3, implying the existence of a low-dimensional manifold in the ictal state, which are called “epileptic attractor”. As such, a value of 7 of the embedding dimension is used to properly reconstruct this epileptic attractor.

Although d of interictal (between seizures) “steady state” EEG data is expected to be higher than that of the ictal state, a constant embedding dimension d=7 has been used to reconstruct all relevant state spaces over the ictal and interictal periods at different brain locations. The advantages of using such a small embedding dimension are that:

a) irrelevant information to the epileptic transition in dimensions higher than 7 does not influence much the values of the dynamical measures, and

b) estimation of the dynamical measures suffers less from the small number of data points allowable per moving window (short windows are used to address the non-stationary feature of the EEG). A disadvantage is that critical information for the transition to seizures that may exist in dimensions higher than 7 may not be captured.

The Lyapunov exponents measure the average information flow (bits/sec) a system produces along local eigenvectors of its movement in its state space. The estimation of the largest Lyapunov exponent (Lₘₐₓ) in a chaotic system is more reliable and reproducible than the estimation of the remaining exponents of the mathematical expression of the signal x(t), especially when the value of d is not known and changes over time, as in the case in high-dimensional and nonstationary data like the interictal EEG. A measure developed to estimate an approximation of Lₘₐₓ from nonstationary data was called STLₘₐₓ (Short-Term maximum Lyapunov exponent). The STLₘₐₓ is estimated from sequential EEG epochs of 10.24 seconds, recorded from electrodes in multiple brain sites, to create a set of STLₘₐₓ profiles over time (resulting to one STLₘₐₓ value per epoch, one STLₘₐₓ profile per recording site) that characterizes the spatio-temporal chaotic signature of the epileptic brain. The STLₘₐₓ profiles at two electrode sites are shown in FIG. 2(c) (left panel). These figures show the evolution of STLₘₐₓ as the brain progresses from interictal to ictal to postictal states. The seizure onset is characterized by a sudden drop in STLₘₐₓ values, with a consequent steep rise in STLₘₐₓ and higher values in the postictal than the ictal period, denoting a chaos-to-order-to-chaos transition. What is also observed is a convergence of the STLₘₐₓ profiles long before the seizure's onset. This convergence is referred to herein as “dynamical entrainment”, because it is progressive (entrainment) and involves measures of the dynamics (dynamical) of the underlying subsystems in the brain. This dynamic entrainment has constituted the basis for the development of the first prospective epileptic seizure prediction algorithms. Hereafter, this dynamic entrainment is referred to as dynamical synchronization or synchronization of the brain dynamics.

T-Index as a Measure for Synchronization of EEG Dynamics

A statistical measure of synchronization between two electrodes i and j, with respect to a measure (e.g. STLₘₐₓ, E or Φₘₐₓ) of their dynamics, has been developed and is described below. Specifically, the Tᵢⱼ between measures at electrode sites i and j and time t is defined as:

$${T}_{ij} ( t ) = \frac{{\hat{D}}_{ij} ( t )}{{\hat{σ}}_{ij} ( t ) / \sqrt{m}}$$

where \hat{D}ᵢⱼ(t) and \hat{σ}ᵢⱼ(t) denote the sample mean and standard deviation respectively of all the m differences Dᵢⱼ(t) between a measure's values (one value per 10.24 sec) at electrodes i and j, within a moving window w₁(t)=[t, t−m*10.24 sec].

If the true mean μᵢⱼ(t) of the differences Dᵢⱼ(t) is equal to zero, and \hat{σ}ᵢⱼ(t) are independent and normally distributed, Tᵢⱼ(t) is asymptotically distributed as the t-distribution with (m−1) degrees of freedom. It has been shown that these independence and normality conditions are satisfied in EEG. Therefore, desynchronization between electrode sites i and j is defined as when (t) is significantly different from zero at a significance level α. The desynchronization condition between the electrode sites i and j, as detected by the paired t-test, is

Tᵢⱼ(t)>t_{α/2,m-1}=Tₜₕ

where t_{α/2,m-1} is the 100·(1−α/2) % critical value of the t-distribution with m−1 degrees of freedom. If Tᵢⱼ(t)≦t_{2,m-1} (which means that there is no satisfactory statistical evidence at the α level that the differences of values of a measure between electrode sites i and j within the time window w₁(t) are not zero), and consider that sites i and j are synchronized with each other (with respect to the utilized measure of synchronization) at time t.

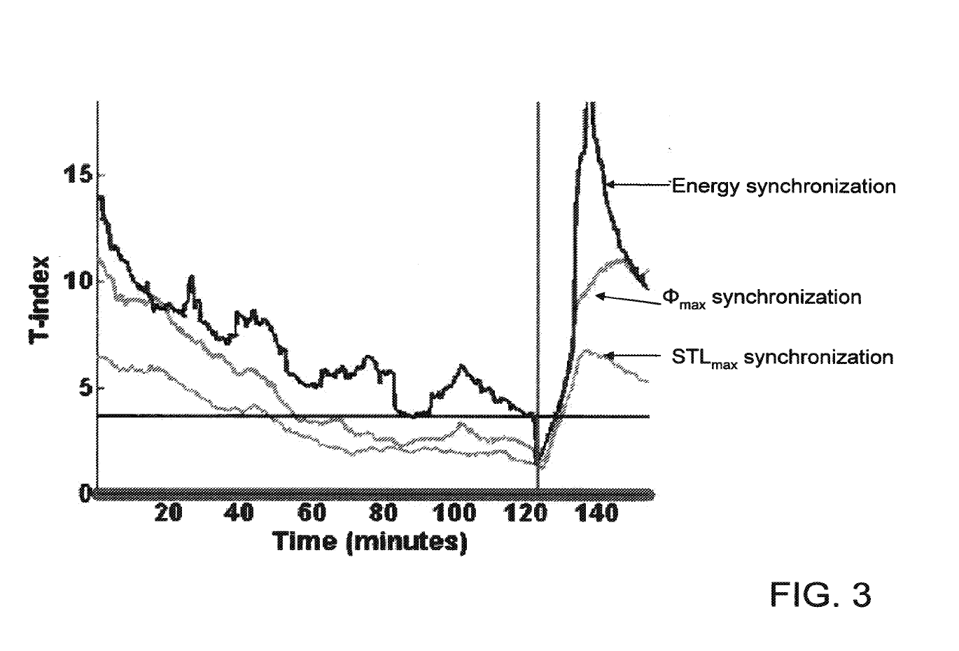

Using α=0.01 and m=60, the threshold Tₜₕ is equal to 2.662. It is noteworthy that similar STLₘₐₓ, E, or Φₘₐₓ values (i.e. when these measures are synchronized) at two electrode sites do not necessarily mean that these sites also interact. However, when there is a progressive convergence (synchronization) over time of the measures at these sites, the probability that they are unrelated diminishes. This is exactly what occurs before seizures, and is illustrated in FIGS. 2 and 3 for all three measures illustrated herein.

In FIG. 2, the T-index profiles are generated from the STLmax profiles of all entrained electrode sites within 10 minutes before each seizure.

A progressive synchronization in all measures, as quantified by Tᵢⱼ(t), is observed preictally. Note that, for each measure, different critical sites are synchronized. On the other hand, if Tᵢⱼ^{t}>t_{α/2,m-1} (which means that there is satisfactory statistical evidence at the α level that the differences of values for a measure between electrode sites i and j within the time window w are not zero), sites i and j are considered to be desynchronized with each other at time t. Using α=0.00001 and m=60, the desynchronization threshold Tₜₕ is equal to 5.0. Then, the divergence of STLₘₐₓ values over time estimated at the two sites is called “dynamical desynchronization.”

Amount/Extent of Spatial Synchronization

In order to explicitly follow the spatial dynamics of electrode sites over time, an amount of synchronization S(t) is defined as the number of pairs of electrodes that are highly synchronized within the time window w₁(t). This amount of synchronization S(t) is then normalized by the total number of electrode pairs available for analysis. Mathematically, the normalized amount of synchronization S(t) is estimated from the T-index matrix T of STLₘₐₓ values at all electrode sites, as:

$$S ( t ) = \frac{card \{ {T}_{ij}^{t} \text{:} {T}_{ij}^{t} \leq {T}_{sth} , T ∈ {R}^{n \times n} , i , j = 1 , … , n \}}{card \{ {T}_{ij}^{t} \text{:} {T}_{ij}^{t} \neq 0 , T ∈ {R}^{n \times n} , i , j = 1 , … , n \}}$$

where the numerator denotes the cardinal number (card) of the set of upper diagonal elements Tᵢⱼ of the T matrix that have values lower than the synchronization threshold Tₛₜₕ, the denominator is the total number of non-zero elements in the T matrix and n refers to the total number of electrodes used for the analysis. Using α=0.01 and degrees of freedom equal to 59, the synchronization threshold Tₛₜₕ is equal to 2.662.

TABLE 2
Mean and standard deviation of seizure predictability time Tₚ of all
entrained site selected per seizure and measure in Patients 1 and 2.
Tₚ (minutes)

| | |
|---|---|
| Patient 1 | Patient 2 |
| (24 seizures) | (19 seizures) |

| Measure | Mean | Std. | Mean | Std. |
|---|---|---|---|---|
| STLmax | 57.45 | 38.50 | 51.69 | 33.62 |
| E | 10.72 | 10.50 | 12.88 | 9.97 |
| Φₘₐₓ | 17.51 | 10.55 | 27.33 | 12.34 |

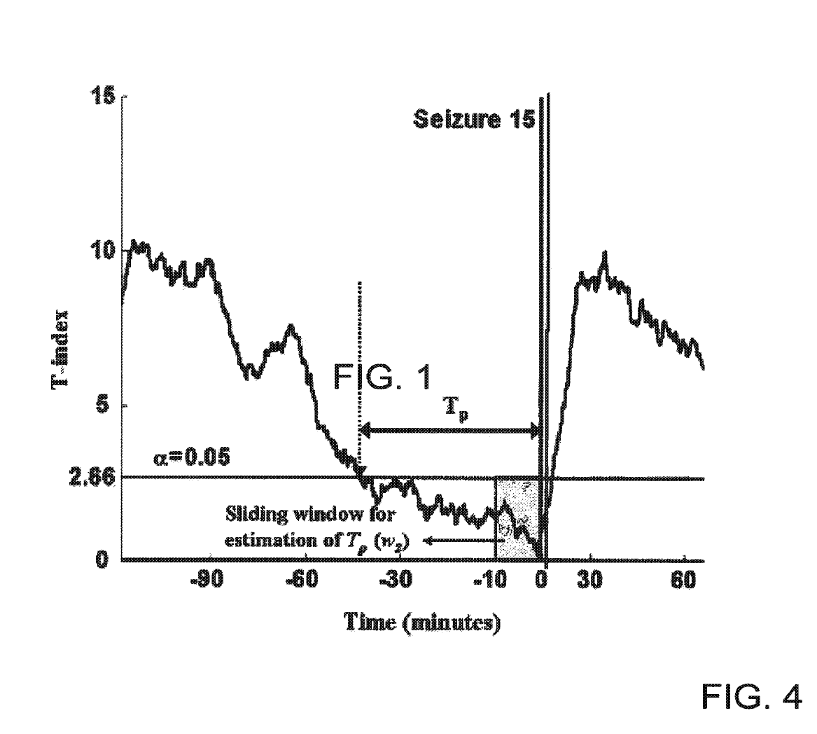

The predictability time Tₚ for a given seizure is defined as the period before a seizure's onset during which synchronization between critical sites remains highly statistically significant (i.e. T-index<2.662). Each measure of synchronization gives a different Tₚ for a seizure. In the estimation of Tₚ, to compensate for possible random oscillations of the T-index profile, average Tₚ values are averaged within windows w₂(t) moving on the T-index profile backward in time from a seizure's onset. A similar length of w₂(t) as the one of w₁(t) may be used so that Tₜₕ is the same for both windows for α=0.01. Then, Tₚ is estimated by the following procedure:
a) The time average T̅-index of the T-indices over 3 hours before a seizure, within w₂(t)=[t, t−10 min], where t*>t>t*−170 min, is continuously estimated until T̅-index is less than or equal to Tₜₕ.
b) When t=t₀: T̅-index>Tₜₕ, the Tₚ is defined as Tₚ=t*−t₀. This predictability time estimation is portrayed in FIG. 4 (where t*=0). The longer the Tₚ, the longer synchronization is observed prior to a seizure.

Dynamical synchronization using STLₘₐₓ consistently resulted in longer predictability times Tₚ than the ones by the other two measures (see Table 2). The phase synchronization measure outperformed the linear, energy-based measure and, for some seizures, it even had comparable performance to that of STLₘₐₓ-based synchronization. These results are consistent with the synchronization observed in coupled non-identical chaotic oscillator models: an increase in coupling between two oscillators initiates generalized synchronization (best detected by STLₘₐₓ), followed by phase synchronization (detected by phase measures), and upon further increase in coupling, amplitude synchronization (detected by energy measures).

Now referring to FIG. 3, a representative example of the behavior of averaged T-index profiles for each measure before Seizure 15 in Patient 1 is shown. This behavior of the T-index profiles is consistently observed in all 43 seizures recorded from these two patients, Patient 1 and Patient 2.

### Seizure Prediction

Prior to the present invention, a seizure-prediction algorithm was developed to be based on the convergence and divergence of STLₘₐₓ among critical electrode sites selected adaptively. A warning of an impending seizure was issued. Global optimization techniques were applied for selecting critical groups of electrode sites.

Two embodiments of improved seizure prediction algorithms are described herein. One algorithm is based on combination of two (among other) measures of synchronization (Algorithm 1) and the other is based on one measure of spatial synchronization of all entrained (synchronized) recording sites (Algorithm 2), which differentiates them from prior algorithms and systems. The algorithms were tested in continuous 5 and 7 days of intracranial EEG recordings respectively from two patients with refractory temporal lobe epilepsy. It is shown that for a 2 hour prediction horizon, Algorithm 1 predicts 82% of all seizures across two patients with an average false prediction rate of 0.12 per hour and Algorithm 2 predicts 83% of seizures with 0.125 false predictions per hour. Seizure warnings were issued on an average of 45.7 minutes and 47.6 minutes before ictal onset by utilizing Algorithm 1 and Algorithm 2 respectively.

The findings of improved predictability via combining dynamical measures, for example phase and STLₘₐₓ, and incorporation of the dynamical synchronization of all entrained pairs of sites have led to incorporating these two features separately and obtaining two new prediction algorithms. For the purpose of this discussion, the prediction algorithm obtained by combining dynamical measures, e.g. phase and STLmax, is referred to as Algorithm 1 and, the one obtained by following the dynamics of all synchronized pairs of sites is referred to as Algorithm 2. The two algorithms consist of the following generic steps:
a) Values of dynamical measures are iteratively calculated from sequential non overlapping 10.24 s EEG epochs obtained from each electrode site. This step accomplishes a large data reduction (each 10.24 s EEG epoch generates one value in the dynamical measure profiles), and is applied to each available EEG channel, creating a new multi-channel time series of dynamical measures utilized in subsequent analysis.
b) In one implementation, all critical electrode pairs at a starting time point are automatically identified and their dynamics are measured and followed forward in time by the algorithm. The critical electrode sites/pairs may be selected as the ones that are entrained first with respect to one measure profiles (for example in the STLmax profiles) prior to a specified starting point of the algorithm.
c) Once all synchronized electrode pairs with respect to their STLₘₐₓ profiles are chosen (critical pairs), their average T-index profile is calculated and monitored forward in time using sequential 10 min sliding windows. In parallel, the average T-index profile from the phase profiles of these electrode pairs is continuously calculated. Both these average T-index values (from the phase and STLₘₐₓ profiles) are continuously compared to a preset statistical threshold value (Tₜₕ), defined as the value below which the average difference of the phase values (as well as of the STLmax values) in the corresponding time window is not significantly different from 0 (p>0.01). When the average T-index from the phase profiles of the critical electrode pairs (i.e. pairs selected as before from the STLₘₐₓ profiles) gets entrained (i.e. initially being>5 (disentrained), drops to a value of 2.662 or less (entrained)—these threshold values were chosen based on statistical considerations because, when the T-index is greater than 5, the average phase or STLmax values for electrode pairs are highly significantly different (p-value<0.00001); when the T-index is less than 2.662, the average phase or STLₘₐₓ values of these electrode pairs are not significantly different (p-value>0.01), a warning of an impending seizure is issued. It is to be understood by those skilled in the art that other combinations of measure profiles could be utilized in this or another order for the selection of the critical pairs to be followed forward in time.
d) After the warning is issued, a user-specified or internal seizure prediction horizon (PH) parameter determines the maximum allowable time the previously found critical pairs would be followed forward in time before the processes of reselection of critical pairs (step b above) and issue of the next warning (step c above) are repeated till the end of the EEG recording.

Embodiments of the proposed invention have been tested to be more reliable in providing early seizure warnings for impending seizures, which can be used to provide improved seizure control. In addition, the present algorithms do not require any patient-specific tuning nor collection of training data to achieve consistent prediction. An embodiment of a method for seizure prediction includes the following:
1) A combination of more than one measure of brain synchronization and the spatial extent of the pathology are utilized to issue more reliable warnings.
2) No complex optimization technique is required to select the critical sites (or pairs).
3) The present method does not require any training of the algorithm on previously recorded data or seizures to specify algorithms' parameters
4) The algorithms can be initiated at any point in time without the need to wait for at least one seizure to occur before it could prospectively run on the data.

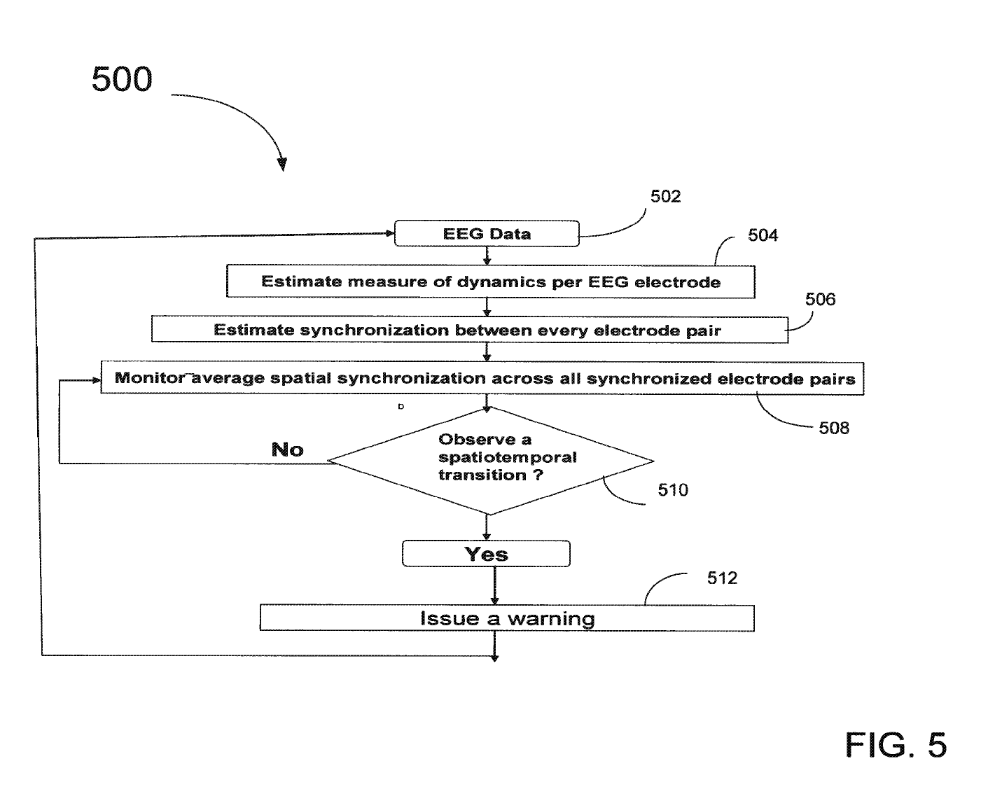

Now referring to FIG. 5, a flow diagram 500 illustrating an embodiment of Algorithm 1 is shown. At step 502, Algorithm 1 is initiated with the acquisition of EEG data. At step 504, dynamic measures are evaluated or estimated for each EEG electrode and synchronization is estimated between every electrode pair in order to determine all critical electrode pairs and follow their dynamics forward in time, at step 506. At step 508, an average spatial synchronization is monitored across all synchronized electrode pairs. Subsequently at step 510, a check is performed as to whether a spatiotemporal transition is detected. In the affirmative, a warning is issued at step 512; otherwise the monitoring across all synchronized electrode pairs is resumed. Notably, the critical electrode sites (or pairs) are automatically selected by Algorithm 1 at any user-specified time t₀ in the EEG record such that they are most synchronized (most converged STLₘₐₓ and/or phase profiles) within a 10-min window prior to t₀.

When the warning is issued (critical sites or pairs synchronized in both their STLmax and phase profiles), new optimal groups of sites are selected by the following procedure:
1) the newly selected groups should be synchronized with respect to STLₘₐₓ in the 10-minute window prior to the warning, and
2) the newly selected groups should be desynchronized in their phase profiles (or desynchronized in both profiles) in the 10-minute window prior to the warning (i.e. the re-selected sites/pairs be disentrained in at least one of the dynamical measures profiles before the reselection point of time t).

In essence, after a warning, the T-indices of the new groups of sites are followed forward in time and the conditions for a warning are applied. Algorithm 1 is very intuitive and aligns well with the theory of dynamical systems wherein, as the coupling between two interacting subsystems is increased, they start to converge in a subspace of their initial joint manifold with their STLmax starting to converge. Following this convergence, the phases of the interacting systems get synchronized. Substantially close to a seizure, amplitude synchronization (i.e. synchronization of energy profiles) of the sites that are already synchronized in STLₘₐₓ and Phase is expected.

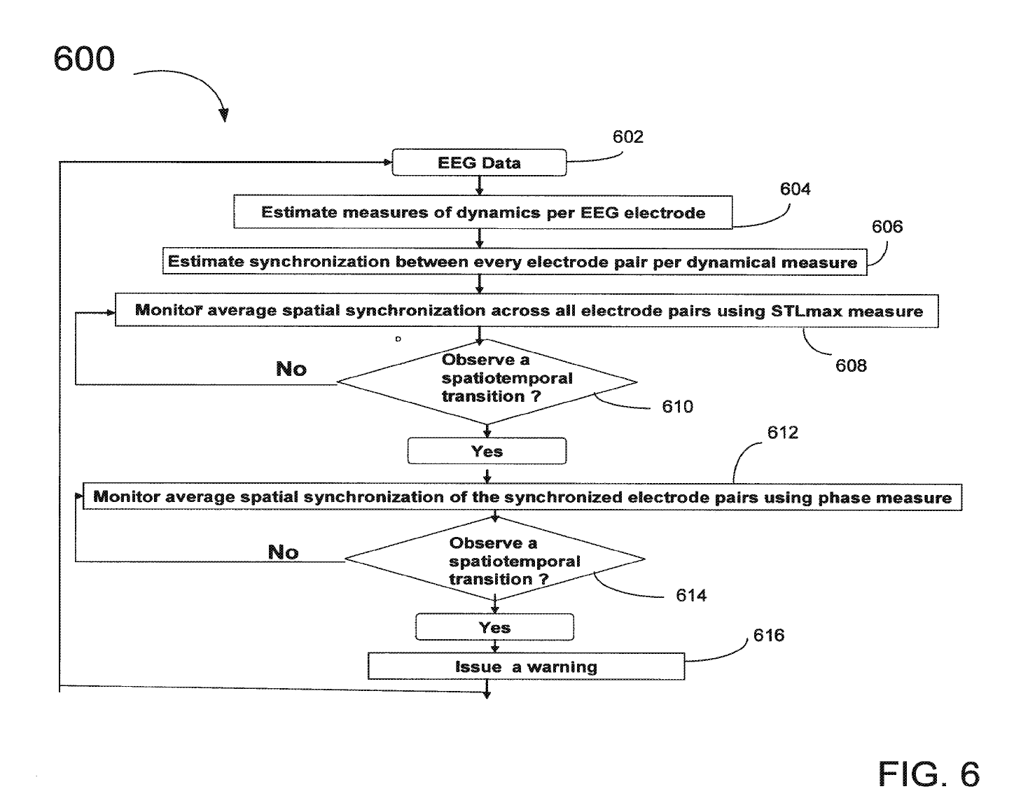

Now referring to FIG. 6, a flow diagram 600 illustrating an embodiment of Algorithm 2 is shown. At step 602, Algorithm 2 is initiated with the acquisition of EEG data. At step 604, dynamic measures are evaluated or estimated for each EEG electrode and synchronization is estimated between every electrode pair in order to determine per dynamiceal measure, at step 606. At step 608, an average spatial synchronization is monitored across all synchronized electrode pairs using the STLmax measure. Subsequently at step 610, a check is performed as to whether a spatiotemporal transition is detected. In the affirmative, another average spatial synchronization is monitored across all synchronized electrode pairs using the phase measure at step 612; otherwise the monitoring across all synchronized electrode pairs using the STLmax measure is repeated. At step 614, another check as to whether another spatiotemporal transition is detected. In the affirmative, a warning is issued at step 516; otherwise the monitoring across all synchronized electrode pairs is using the phase measure is resumed.

Similarly to Algorithm 1, the initial set of electrode pairs that are followed in Algorithm 2 can be selected at any user-specified time point in the entire record and consists of all pairs of sites that are entrained (converged in STLmax profiles) within a 10-minute window prior to this time point. Once all entrained electrode pairs are chosen, the average T-index profile of all entrained pairs is continuously calculated from the STLmax profiles of the sites, using sequential 10 min sliding windows forward in time. The average T-index values are continuously compared to a preset threshold value (Tₜₕ), defined as the value below which the average difference of STLmax values in the corresponding time window is not significantly different from 0 (p>0.01). When the average T-index of the selected pairs of sites becomes less than Tₜₕ, the pairs are considered to be synchronized and a warning is issued.

Algorithm 1 was tested on Patient 1 and Patient 2, and the results are summarized in Table 3.

Under this condition, the percentage of clinical seizures that were correctly predicted ranged from 89% (Patient 1) to 91% (Patient 2), with an average of 90.63% sensitivity across both patients. Recordings obtained in Patient 2 included ictal EEG discharges without observed or reported clinical symptomatology (subclinical seizures). The algorithm correctly predicted 55.56% ( 5/9) of the subclinical seizures in Patient 2. On average, the algorithm generated a prediction approximately 47 minutes before each seizure, estimated as the average of the distance in time of the true predictions from the subsequent seizure onset. Under this condition of fixed detection parameters, the false predictions occurred at a rate ranging from 0.09 to 0.15 (mean 0.12) false predictions per hour. This on average corresponds to a false warning every 8.33 hours.

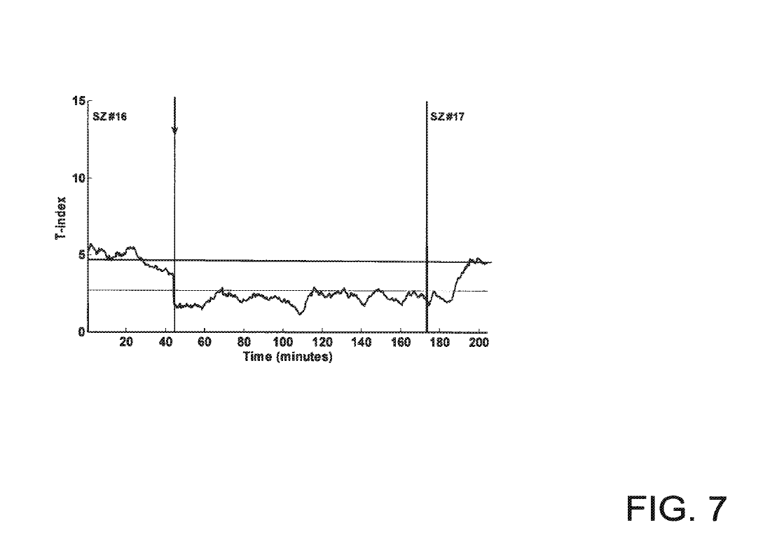

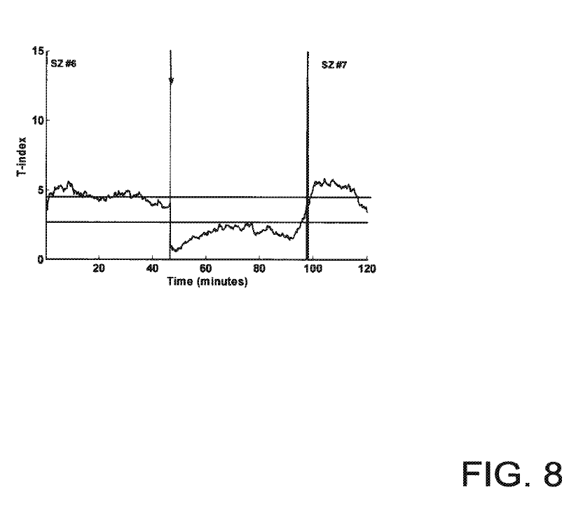

In FIGS. 7 and 8, prediction results from Algorithm 1 for some seizures recorded from Patients 1 and 2 are shown respectively. In particular, the times of the warnings issued by Algorithm 1 along with the corresponding T-index curves are shown. From these figures, the preictal trends of sites are followed by Algorithm 1 to the upcoming seizure. Moreover, the sites followed in Algorithm 1 stay synchronized up to the seizure following which they get desynchronized. This behavior is very consistent for most of the seizures predicted by Algorithm 1.

TABLE 3
PERFORMANCE OF ALGORITHM 1 FOR PATIENT 1 AND PATIENT 2.

| | Sensitivity (%) | | | False Positive | Mean Prediction |
|---|---|---|---|---|---|
| Patient | Clinical | Sub-clinical | Overall | rate (per hour) | time (min.) |
| Patient 1 | 21/23 | — | 21/23 | 0.09 | 48.7 ± 7.6 |
| Patient 2 | 8/9 | 5/9 | 13/18 | 0.15 | 45.5 ± 9.2 |
| Total | 29/32 | 5/9 | 34/41 | 0.12 | 47.12 ± 8.4 |
| | (90.63%) | (55.56%) | (82.93%) | | |

Algorithm 2 was tested in Patient 1 and Patient 2. These results are summarized in Table 4. For Algorithm 2, the percentage of clinical seizures that were correctly predicted ranged from 86% (patient 1) to 89% (patient 2), with an average of 87.50% sensitivity overall. In patient 2, the algorithm correctly predicted 55.56% ( 5/9) of the subclinical seizures, identical to the results obtained from Algorithm 1. On average, the algorithm generated a prediction approximately 48 minutes before each seizure, estimated as the average of the distance in time of the true predictions from the subsequent seizure onset. The false predictions occurred at a rate ranging from 0.09 to 0.12 (mean 0.105) false predictions per hour, a marginal improvement over the false prediction rate of Algorithm 1. This on average corresponds to a false warning every 9.52 hours.

TABLE 4
PERFORMANCE OF ALGORITHM 2 FOR PATIENT 1 AND PATIENT 2

| | Sensitivity (%) | | | False Positive | Mean Prediction |
|---|---|---|---|---|---|
| Patient | Clinical | Sub-clinical | Overall | rate (per hour) | time (min.) |
| Patient 1 | 20/23 | — | 20/23 | 0.09 | 49.2 ± 7.2 |
| Patient 2 | 8/9 | 5/9 | 13/18 | 0.12 | 47.5 ± 8.2 |
| Total | 28/32 | 5/9 | 33/41 | 0.105 | 48.35 ± 7.7 |
| | (87.50%) | (55.56%) | (80.49%) | | |

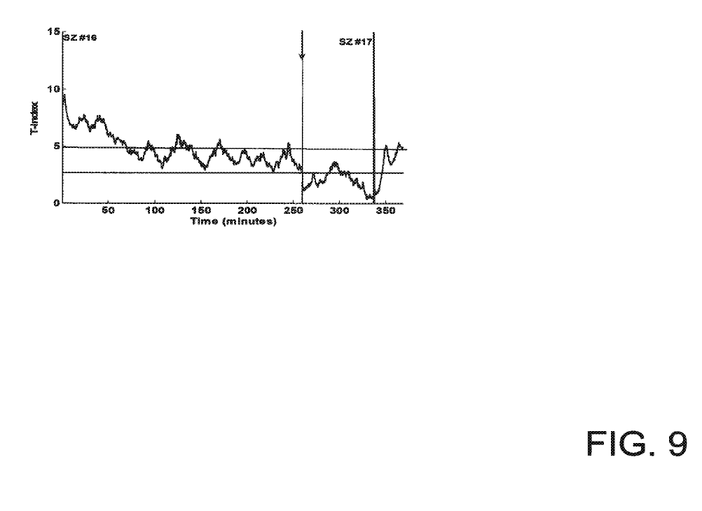

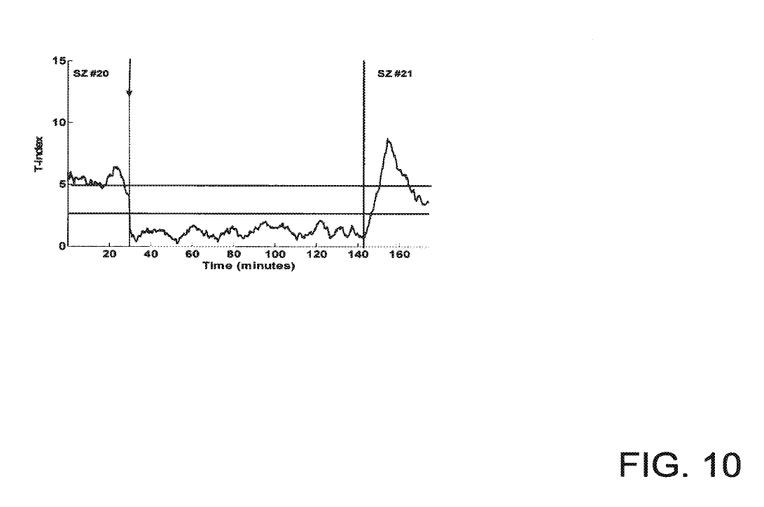

Seizure prediction performance results of Algorithm 1 and Algorithm 2 are summarized in Table 5, and dynamical transitions issued by Algorithm 1 and Algorithm 2 are illustrated in FIGS. 9-10.

TABLE 5
PREDICTION PERFORMANCE OF ALGORITHM 1 AND ALGORITHM 2

| | Sensitivity (%) | | False Positive rate (per hour) | | Mean Prediction time (min.) | |
|---|---|---|---|---|---|---|
| Patient | Algorithm 1 | Algorithm 2 | Algorithm 1 | Algorithm 2 | Algorithm 1 | Algorithm 2 |
| Patient 1 | 21/23 | 20/23 | 0.09 | 0.09 | 48.7 ± 7.6 | 49.2 ± 7.2 |
| Patient 2 | 13/18 | 13/18 | 0.15 | 0.12 | 45.5 ± 9.2 | 47.5 ± 8.2 |
| Total | 34/41 | 33/41 | 0.12 | 0.105 | 47.12 ± 8.4 | 48.35 ± 7.7 |
| | (82.93%) | (80.49%) | | | | |

Seizure Detection

An embodiment of a method for detecting seizures combines the spatial extent and level of dynamical synchronization of two or more measures of brain synchronization to achieve reliable automated seizure detection, and comprises the following steps:
a) Values of Lyapunov exponent, Phase and Energy measures are iteratively calculated from sequential non-overlapping 10.24 sec EEG epochs per electrode site. This step accomplishes a large data reduction (each 10.24 s EEG epoch generates a single value in the dynamical measure profiles), and it is applied sequentially to each EEG channel, creating a new multi-channel time series that is utilized for subsequent analysis.
b) Dynamical measures of synchronization are iteratively estimated from sequential, 1-point overlapping, 10-minute windows over the measure profiles, between every pair of phase as well as energy profiles, thus generating dynamical phase and energy synchronization profiles
c) A measure of amount/extent of spatial synchronization is estimated per electrode per 10 minute epoch combining the two dynamical synchronization profiles
d) A threshold for seizure detection is determined through cross-validation
e) A seizure is detected when the estimated measure of spatial synchronization reaches values above the preset threshold.

The amount of entrainment/synchronization (PSP(t)-portion of synchronized pairs) at time t is quantified as the number of pairs that remain significantly synchronized (i.e. within a 10 minute window immediately before time t) in both their measures of phase and energy divided by the total number of available pairs. This quantity, a measure of the spatial extent of the critical sites, is large at the seizure onset. The measures of PSP are continuously calculated over time in the entire EEG data recorded from two patients. A cut-off threshold is determined via cross-validation in order to maximize the seizure detection performance. This threshold is then used to detect seizures in these patients.

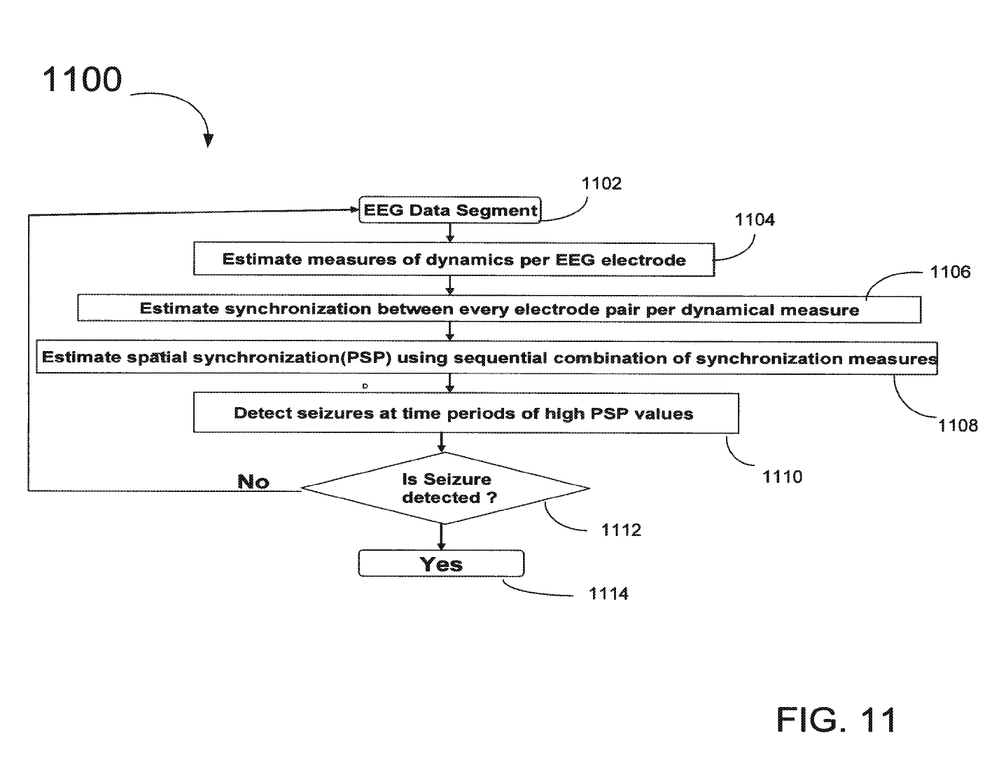

Now referring to FIG. 11, a flow diagram 1100 illustrating an embodiment of a seizure detection algorithm is shown. At step 1102, the algorithm is initiated with the acquisition of EEG data. At step 1104, dynamic measures are evaluated or estimated for each EEG electrode and synchronization is estimated between every electrode pair in order to determine per dynamical measure, at step 1106. At step 1108, a spatial synchronization (PSP) is monitored using a sequential combination of synchronization measures. Subsequently at step 1110, a monitoring for seizures at tile periods of high PSP values is performed. A check is performed as to whether a seizure is detected, at step 1112. In the affirmative, the seizure detection is recorded at step 1114; otherwise the acquisition of EEG data is resumed,

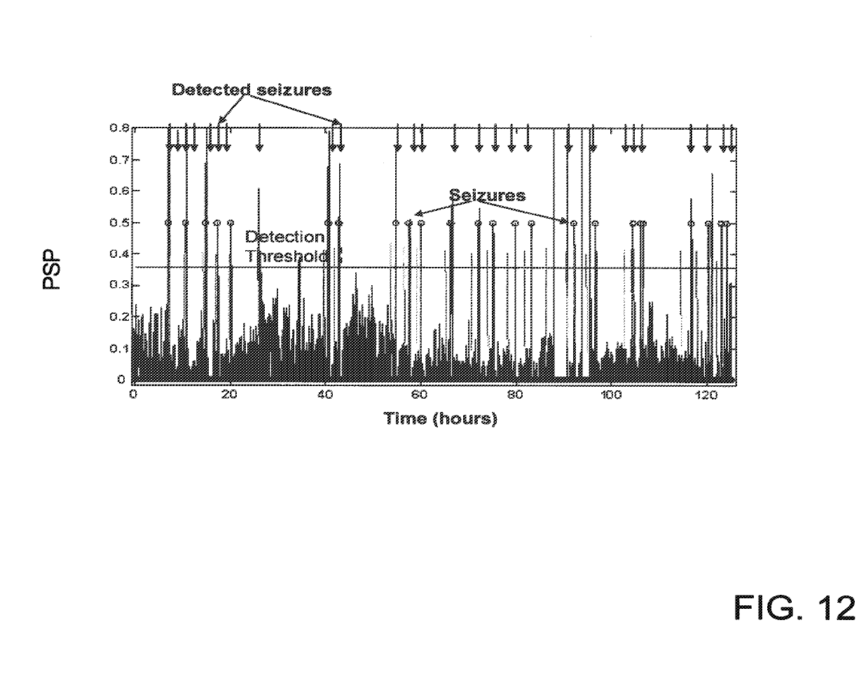

Now referring to FIG. 12, the seizure detection applied to 5 days EEG data from Patient 1, who experienced 24 seizures, is shown. Vertical lines denote the actual seizures that may be marked by a physician, for example, and vertical arrowed lines denote the ones marked by the algorithm. The horizontal line denotes the threshold that was used for this particular data set. From FIG. 12, it is clear that the algorithm is able to track all the seizures that were marked by the physicians and a few more. It is desirable to determine whether some of these detections, which are in addition to the ones marked by the physician, actually correspond to missed seizures or algorithmic error.

An epileptogenic focus is defined electrophysiologically as the area in the brain that is the major source of interictal epileptiform EEG discharges (spikes) and exhibits the earliest onset of epileptic seizures (ictal onset). EEG epileptiform discharges are either focal (single epileptogenic focus), multifocal (independent epileptogenic foci) or diffuse (no definite epileptogenic focus). Detailed electrophysiological investigations of patients who clinically appear to have a single well-localized epileptogenic lesion may reveal the existence of multiple sources of interictal epileptiform EEG discharges and occasionally more than one site of ictal onset (multiple foci). It is also known that the epileptogenic focus is sometimes transient and shifting from one area of the brain to another. A primary epileptogenic focus may give rise to secondary epileptogenic foci. EEG signals are very helpful in providing evidence for a partial (focal) seizure disorder but, due to the shifting and multiplicity, they are often not reliable in visually identifying the primary epileptogenic focus.

The primary objective in presurgical evaluation is to identify the region which is most responsible for generating the patient's habitual seizures. Usually, resection of this brain tissue is sufficient to abolish epileptic seizures in carefully selected unifocal patients. The epileptogenic focus is generally found from long term monitoring of EEG and localization of seizures' onset. Epileptogenic focus localization using analytical signal processing techniques of the EEG records has profound importance in surgery and epilepsy treatment.

The hypothesis considered herein is that the epileptogenic focus entrains more and more electrodes into a critical mass during the preictal period that eventually leads to a seizure. Therefore, it is expected that the study of spatial distribution of sites that are entrained during the precital period will help us understand the behavior of the focus. Specifically, the present goal is directed to investigating the interictal and preictal changes that occur in the connections of the epileptogenic focus with itself as well as with other electrode sites.

An embodiment of a method for localizing the epileptogenic focus by dynamical analysis of the EEG comprises the following general steps:
a) Values of a dynamical measure are iteratively calculated from sequential non-overlapping, 10.24 sec EEG epochs obtained from each electrode site. This step accomplishes a large data reduction (each 10.24 s EEG epoch generates one value in the dynamical measure profiles), and it is applied to each available EEG channel, creating a new multi-channel time series utilized for subsequent analysis.
b) Values of synchronization are iteratively estimated, from sequential 1-point overlapping, 10-minute windows over the measure profiles, generating one value per pair of measure profiles every 10.24 sec.
c) The amount/extent of spatial synchronization is estimated per electrode over 10-minute epochs in the dynamical synchronization profiles
d) Focus is identified as the electrode/area or a set of electrodes/areas with the largest amount of synchronization for a length of time (e.g. 30 minutes).

Advantageously, the system is capable of detecting areas of abnormal epileptic brain activity days before the occurrence of a seizure, and therefore, providing early seizure localization information from EEG. Hence, this system is substantially cost-effective and accessible.

It has been found that seizures are not abrupt transitions in and out of an abnormal ictal state (seizure), instead they follow a dynamical transition that evolves over minutes to hours. During this preictal dynamical transition, multiple regions of the cerebral cortex progressively approach a similar dynamical state. The dynamics of the preictal transition are highly complex. Even in the same patient, the participating cortical regions and the duration of the transition may vary from seizure to seizure.

During the seizure (ictal state), widespread cortical areas make an abrupt transition to a more ordered state. After the seizure, brain dynamics revert to a more disordered state in which previously entrained (synchronized) cortical areas become disentrained (desynchronized-postictal state). The epileptic brain repeats this series of state transitions intermittently, at seemingly irregular but, in fact, time-dependent intervals. This implies that the transition into seizures is not a random process. Based on the analysis of a small sample of seizure recordings, seizures may serve as intrinsic mechanisms to desynchronize brain areas that were dynamically synchronized in the immediate preictal periods. This phenomenon has been defined as “resetting” of the epileptic brain via recurrent seizures.

Elucidation of the mechanisms of this resetting process may be explained by the following three characteristics:
a) A Level of Resetting (LR) is quantified by the difference of the average T-index value at critical sites between 10 min before (immediate preictal state) and 10 min after (immediate postictal state) a seizure's onset;
b) A Rate of Resetting (RR) is quantified by the ratio of the time it takes the critical sites to disentrain postictally (desynchronization period) over the time they were entrained preictally (synchronization period). This ratio is equivalent to the estimation of the ratio of the slope of the T-index curve in the postictal over the one in the preictal period, because the synchronization/desynchronization threshold for the T-index values is the same (Tₜₕ=2.662)−the end points of the synchronization and desynchronization periods correspond to the same T-index value of Tₜₕ;
c) the amount of entrainment/synchronization PSP(t) at time t defined as the amount of pairs that remain significantly entrained immediately before time t (i.e. in the immediately 10 minute window immediately preceding time t).

Using these three metrics of resetting, it has been demonstrated that brain resetting of dynamically synchronized cortical sites, as measured by LR, RR or PSP, occurs much more frequently at seizure points than at randomly selected points in the available interictal EEG recordings per patient. The idea of resetting may be extended to propose that the therapeutic effect of any seizure control strategy/treatment modality is due to a resetting of the dynamics of the epileptic brain's electrical activity. This resetting is characterized by short and/or long-term disentrainment (desynchronization) of critical brain sites with the epileptogenic focus (foci). This resetting produced by different seizure control strategies may be measured via the aforementioned metrics of LR, RR and PSP to assess the corresponding short-term and long-term control efficacy.

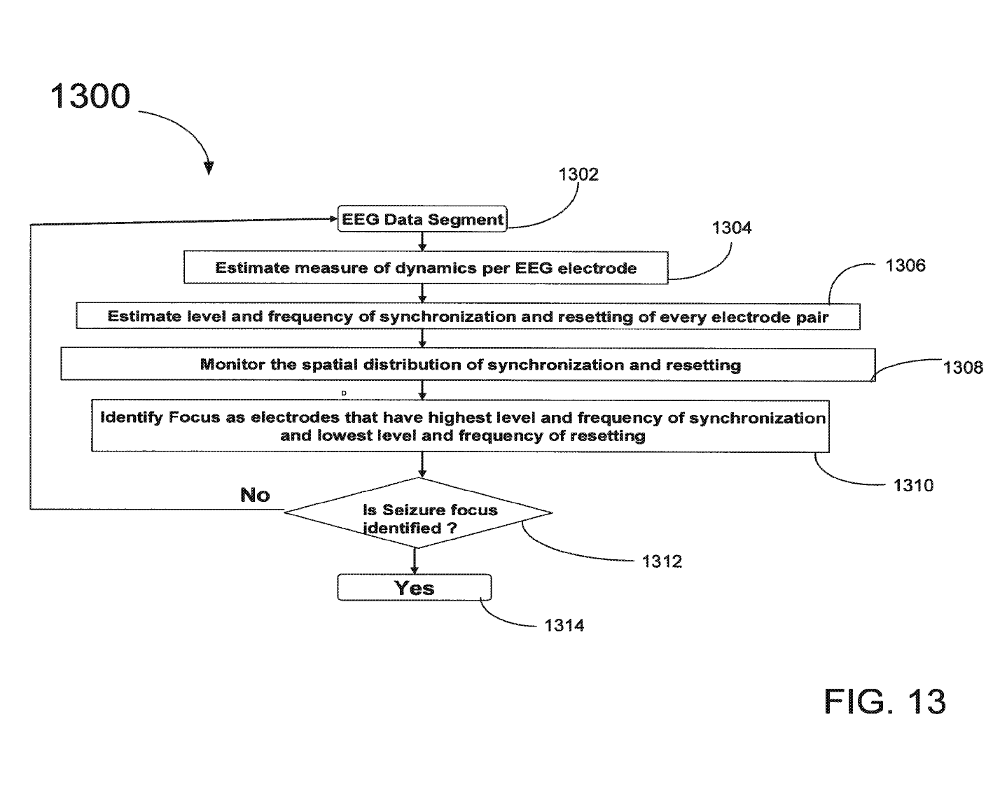

Now referring to FIG. 13, a flow diagram 1300 illustrating an embodiment of a method for determining seizure focus localization is shown. At step 1302, the method is initiated with the acquisition of an EEG data segment. At step 1304, dynamic measures are evaluated or estimated for each EEG electrode. A level and frequency of synchronization and resetting of every electrode pair are estimated, at step 1306. At step 1308, a spatial distribution of synchronization and resetting is monitored. Subsequently at step 1310, electrodes that have highest level and frequency of synchronization and lowest level and frequency of resetting are determined thereby identifying a location of the seizure focus. A check is performed as to whether a seizure is detected or identified, at step 1312. In the affirmative, the seizure detection is recorded at step 1314; otherwise the acquisition of another EEG data is resumed.

For a brain area i as reference, a window w(t) ε [t, t−10] minutes (that is of length of 60 STLₘₐₓ points), the probability of an area A interacting with an area B is defined as follows:

$${P}_{A - B} = \frac{1}{ab} {\sum }_{i = 1}^{a} {\sum }_{j = 1}^{b} Θ ( {T}_{ij}^{t} < {t}_{α / 2 , m - 1} )$$

where α is the number of electrodes in area A, b is the number of electrodes in area B, Θ is the Heaviside step function i.e. Θ(s>0)=1 and Θ(s<0)=0. One P value is then estimated per area quantifying the average number of synchronized electrode pair interactions within this area and with other areas (inclusion of both within and across-area interactions). Subsequently, the P of sites that are continuously entrained for at least 30 minutes before each time instant t is also estimated.

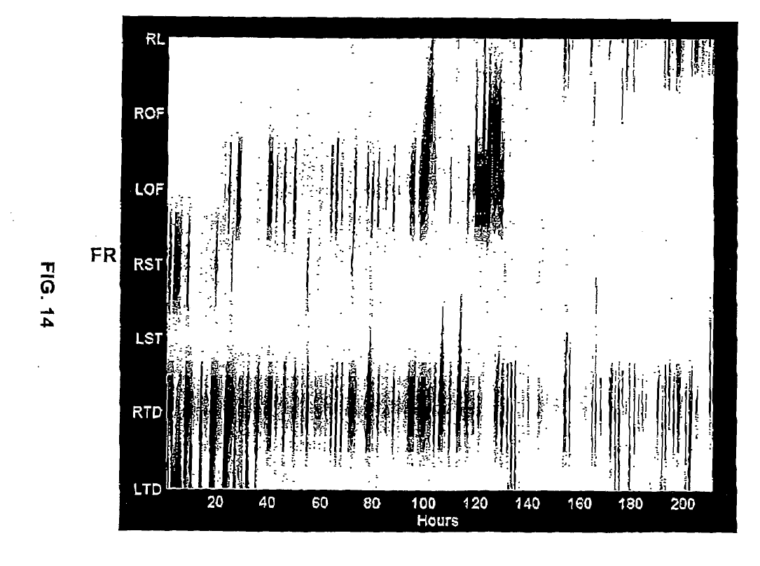

Now referring to FIG. 14, the spatial distribution per area of sites is selected as being entrained (S) and sites that are entrained for at least a time period of 30 minutes (Sᵣᵣ) respectively for Patient 1. From the figures, increased levels of synchronization are observed in both S (increase in the values of S) and Sᵣᵣ (increase in the values of Sᵣᵣ) profiles of the focal area RTD throughout the record. However, this increased level of spatial synchronization at the focus is more pronounced when the criterion of duration of entrainment is taken into account. This observation is consistent across all patients analyzed. From these results, it appears that the focal area remains active throughout a long record. This study also provides a method for identifying the epileptogenic focus, long before the occurrence of the first seizure.

For the purpose of illustration and not limitation, a method to objectively measure the efficacy of any kind of electrical/magnetic/drug-based seizure control scheme is presented and accordingly determines the need for a subsequent one using the analysis of EEG, and comprises of the following steps:
a) Values of a dynamical measure are iteratively calculated from sequential non-overlapping 10.24 s EEG epochs obtained from each electrode site. This step accomplishes a large data reduction (each 10.24 s EEG epoch generates a single value in the dynamical measure profiles), and it is applied sequentially to each EEG channel, thus creating a new multi-channel time series utilized in subsequent analysis.
b) Dynamical measures of synchronization are iteratively estimated, from sequential, 1-point overlapping, 10-minute windows on every pair of measure profiles, thus generating dynamical synchronization profiles
c) A global measure of amount/extent of spatial synchronization and resetting is estimated from the entire set of the dynamical synchronization profiles
d) A short-term successful seizure control is achieved when the applied seizure control strategy resets the observed abnormal synchronization i.e. when the amount of synchronization is significantly reduced during the immediate post treatment period as compared to before treatment.
e) A long-term successful seizure control is achieved when the applied seizure control strategy resets the observed abnormal synchronization for a longer period of time. This is manifested as a progressive reduction in the amount of synchronization over a long period of time.

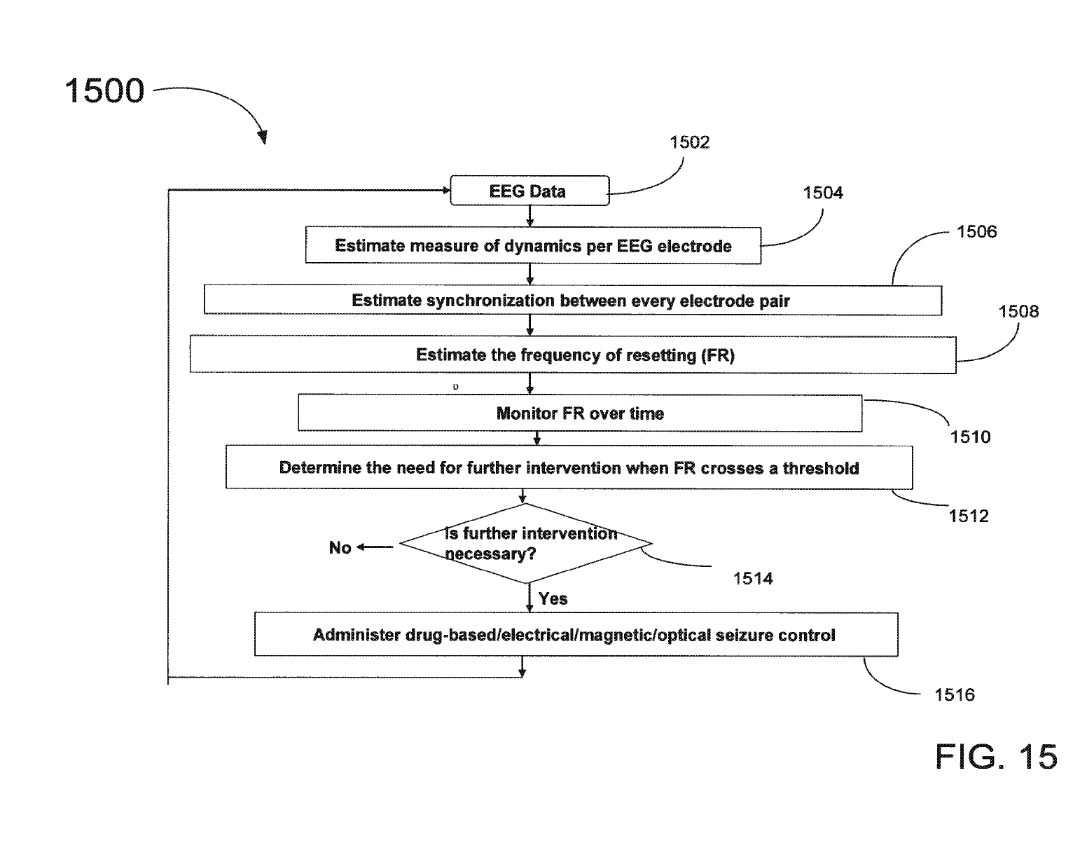

Now referring to FIG. 15, a flow diagram 1500 illustrating an embodiment of a method for evaluating seizure control efficacy is shown. At step 1502, the method is initiated with the acquisition of an EEG data. At step 1504, dynamic measures are evaluated or estimated for each EEG electrode. Synchronization between every electrode pair are estimated, at step 1506. At step 1508, a frequency of resetting FR is estimated, and subsequently monitored over time, at step 1510. When FR is detected to have crossed a predetermined threshold, at step 1512, a check is performed as to whether a seizure controlling intervention is necessary, at step 1514. In the affirmative, the seizure controlling intervention is initiated by administering a drug-based, electrical, magnetic or optical therapy to the patient, at step 1516. In order to track the efficacy of the administered seizure controlling intervention, the acquisition of EEG data is resumed to maintain the monitoring of FR forward in time.

Currently, there exists no tool that quantifies the short-term and long-term efficacy of any kind of seizure control. The existing techniques make use of subjective variables such as the patients' seizure diaries, family member's opinions which can be easily biased and sometimes, incomplete.

For the purpose of illustration and not limitation, the amount of entrainment/synchronization (PSP) may be used as a dynamical measure of synchronization to correlate the success and failure of a feedback control scheme.

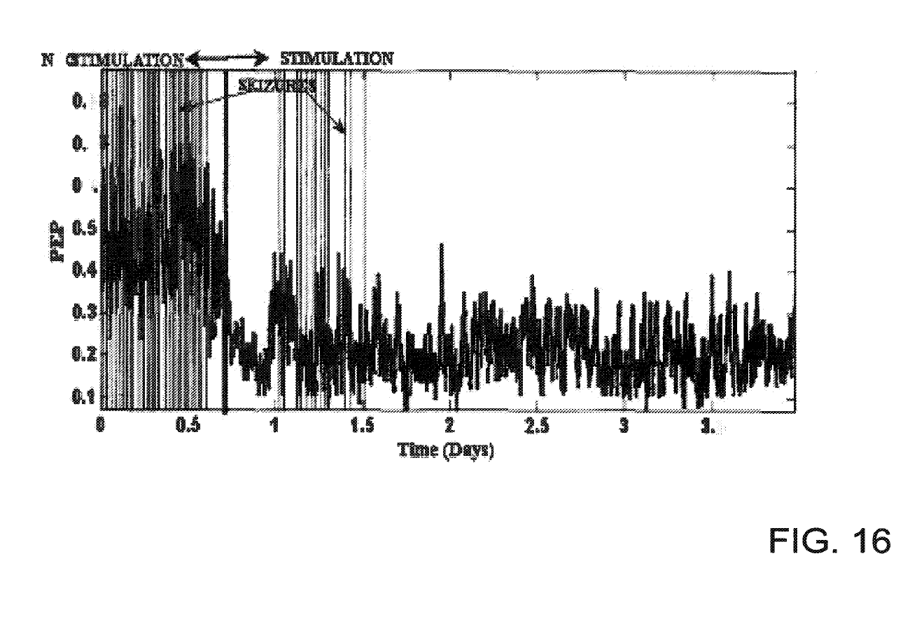

The measures of PSP were continuously calculated over time in the entire EEG data recorded from epileptic rats. There is a significant decrease in spatial synchronization when the warning-based control is effective (from days 2 to 4 in FIG. 16) whereas the spatial synchronization is high during the days when the rat is not given any external stimulation.

The results of the spatial distribution of synchronization show that it may be used to improve seizure control. For instance, the level of spatial synchronization could be combined with the level of entrainment and monitored over time to check when the control scheme starts to become ineffective. Consequently, critical sites that have to be controlled in order to abort subsequent seizures may be identified.

Status epilepticus (SE), which is defined as recurrent epileptic seizures without recovery of normal function between seizures, is the most serious complication of epilepsy. It is a medical emergency, because repeated uncontrolled seizures are life threatening, so status epilepticus must be stopped as quickly as possible. Any kind of epileptic seizure, if repeated frequently enough or prolonged enough, can be considered status epilepticus. However, the most serious and life-threatening form of status epilepticus is generalized convulsive status epilepticus (GCSE), which is by far the most common form of SE. GCSE may be overt, where a patient has a series of generalized convulsions without full recovery between the convulsions, or subtle, where the convulsive activity is markedly attenuated, but the patient is in profound coma because of ongoing electrical seizure activity in the brain. Both presentations of GCSE are serious life-threatening conditions. Death within 30 days after an episode of overt GCSE occurs in 20-26% of affected patients (total of 384 patients suffering from overt GCSE were included in the study), while 65% of patients after subtle GCSE (total of 134 patients suffering from subtle GCSE were included in the study).

Mortality is due to both the underlying condition that caused the episode of GCSE, and also to the devastating encephalopathogenic effects of prolonged seizure activity. The best way to prevent such damaging effects of continuing seizure activity is to rapidly diagnose SE and initiate vigorous therapeutic efforts (antiepileptic drugs) to stop ongoing seizure activity. This is especially true because there is now abundant evidence that the longer SE continues the harder it is to stop with antiepileptic drugs in both humans and animals.

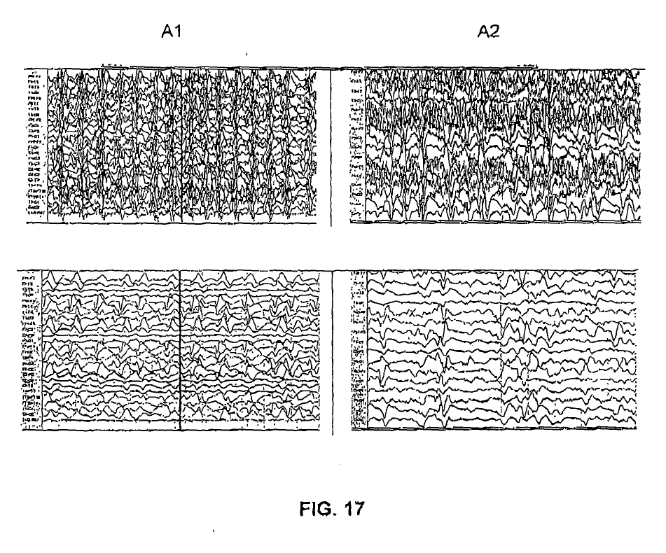

In usual clinical situations, where only limited amount of EEG is stored and viewed, it becomes a challenge for the electroencephalographer to visually differentiate between different diagnoses that occur with morphologically similar patterns on the EEG, no matter how experienced the electroencephalographer might be at reading SE EEGs. The most challenging morphology that has been reported is the occurrence of rhythmic and periodic patterns of uncertain significance. Guidelines for EEG diagnosis of SE have been suggested, and recently an American Clinical Neurophysiology Society committee published a proposal for standardized terminology for rhythmic and periodic EEG patterns in critically ill patients. Unfortunately, however, the first attempt to test this protocol met with significant difficulty. Five board-certified electroencephalographers at the Barrow Neurological Institute reviewed samples of 59 EEGs recorded during SE. There were no samples for which all five observers agreed across all variables. Substantial agreement beyond chance (66.1%) was seen only for determination of whether a sample pattern was rhythmic/periodic, even among five electroencephalographers at the same institution. FIG. 17 shows the morphological similarity between an EEG recorded during subtle GCSE and one recorded in a patient with metabolic encephalopathy (ME).

These figures shed light on the difficulty in correctly differentiating a case of SE from that of ME where morphologically similar EEG patterns visually occurring in both disorders at different time points in the record.

In another embodiment, a method provides a diagnostic tool that distinguishes the condition of Status Epilepticus from other brain disorders such as different types of encephalopathy, non-epileptic seizures, and so forth. This diagnostic method comprises of the following steps:
a) Values of a dynamical measure are iteratively calculated from sequential non-overlapping 10.24 s EEG epochs obtained from each electrode site. This step accomplishes a large data reduction (each 10.24 s EEG epoch generates a single value in the dynamical measure profiles), and it is applied sequentially to each EEG channel, thus creating a new multi-channel time series utilized in subsequent analysis.
b) Dynamical measures of synchronization are iteratively estimated, from sequential, 1-point overlapping, 10-minute windows on every pair of measure profiles, thus generating dynamical synchronization profiles
c) A global measure of amount/extent of spatial synchronization and resetting is estimated from the entire set of the dynamical synchronization profiles
d) A diagnosis of SE is made when the amount of synchronization is of significantly high values. On the other hand, an episode of encephalopathy is accompanied by lower values of amount of synchronization, approximating baseline values.

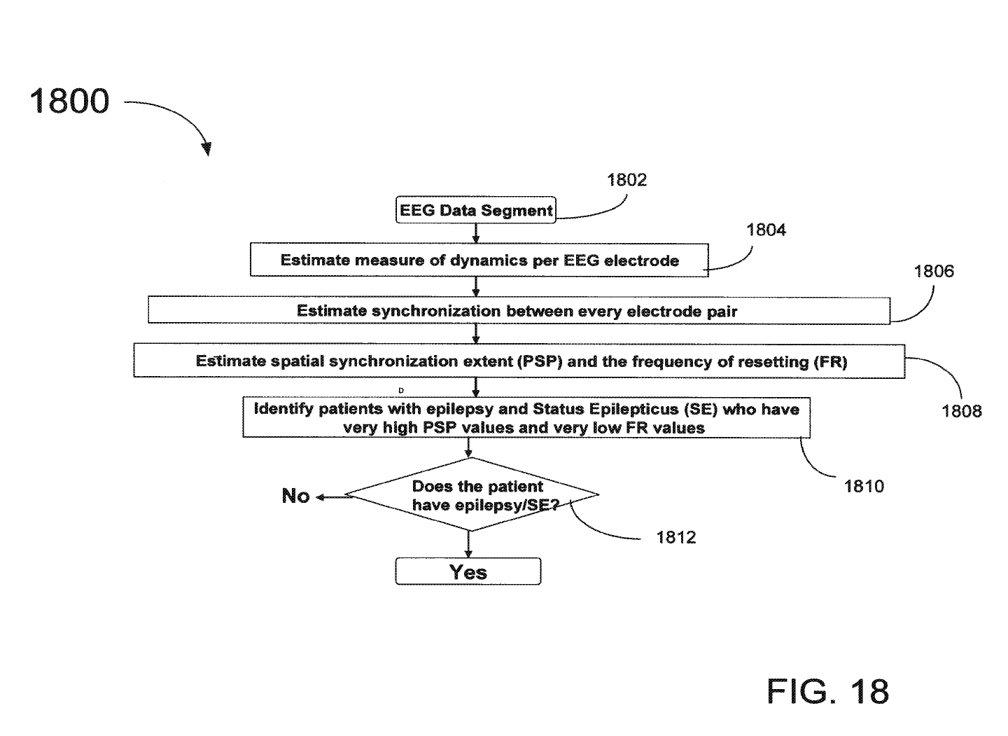

Now referring to FIG. 18, a flow diagram 1800 illustrating an embodiment of a method for distinguishing a condition of Status Epilepticus from other brain disorders is shown. At step 1802, the method is initiated with the acquisition of an EEG data segment. At step 1804, dynamic measures are evaluated or estimated for each EEG electrode, and synchronization between every electrode pair is estimated, at step 1806. At step 1808, a spatial synchronization extent (PSP) and the frequency of resetting FR are estimated. Subsequently, a combination of high PSP values and low FR values is used to identify the patient with epilepsy and SE, at step 1810. Based on this identification, a check is performed as to whether the identified patient does have epilepsy and SE, at step 1812.

Currently, there is no scheme of objective diagnosis of Status Epilepticus available for use in the market. The physicians still rely on visual inspection of the EEG to make their final assessment about such a life-threatening condition. A reliable neuro-diagnostic monitoring tool for Status Epilepticus may supplement a physician's clinical decisions to achieve a more consistent diagnosis of SE which eventually will lead to a better treatment outcome.

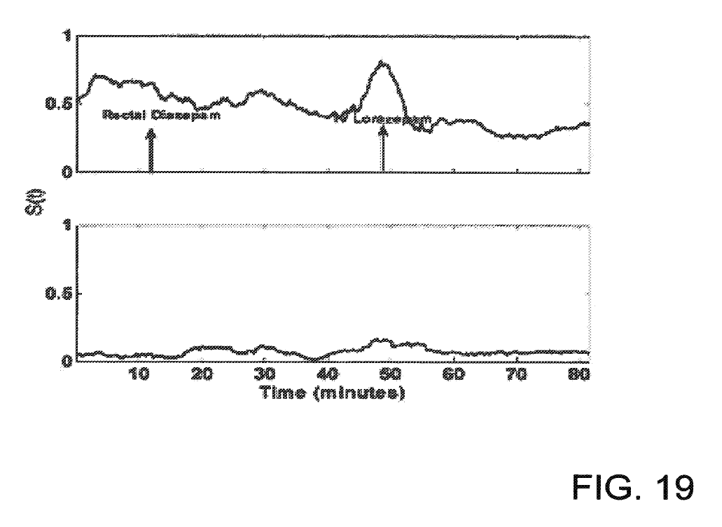

Now referring to FIG. 19, it is clear that S (t) values are significantly higher in the SE data than in the ME data for the entire record. The S (t) values for the SE data start at a value of 0.75, where the patient is in status, and reduce to a value of 0.35 by the end of the record, where the patient recovers. For the ME data, the S (t) values stay at very low values of 0.1 for most of the time. Assuming the hypothesis that SE has a more severe pathology than ME, the brain can be at a higher level of spatial entrainment in SE than in ME, and use this observation as a feature to distinguish between the two conditions.

As stated above, the ability to predict epileptic seizures prior to their occurrences may be beneficial to improving diagnostic tools and treatment of epilepsy. Moreover, an advantageous method based on the above discussed resetting metrics of the multi-component system, namely FR, LR and PSP, may enable a determination of the patient's susceptibility to epileptic seizures.

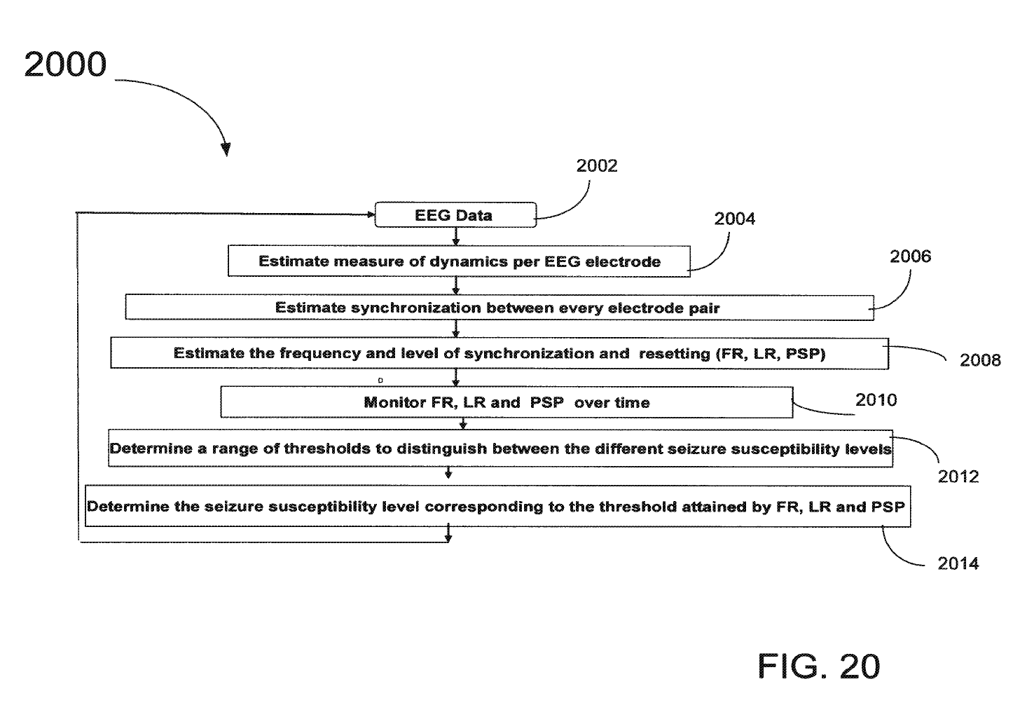

Now referring to FIG. 20, a flow diagram 2000 illustrating an embodiment of a method for determining seizure susceptibility is shown. At step 2002, the method is initiated with the acquisition of an EEG data. At step 2004, dynamic measures are evaluated or estimated for each EEG electrode, and synchronization between every electrode pair is estimated, at step 2006. At step 2008, the frequency of resetting FR, the level of resetting LR and the spatial synchronization extent (PSP) are estimated, and monitored over time at step 2010. Subsequently, a range of thresholds to distinguish between different seizure susceptibility levels is determined, at step 2012. Based on the range determination, the seizure susceptibility level corresponding to the thresholds attained by FR, LR and PSP, at step 2014.

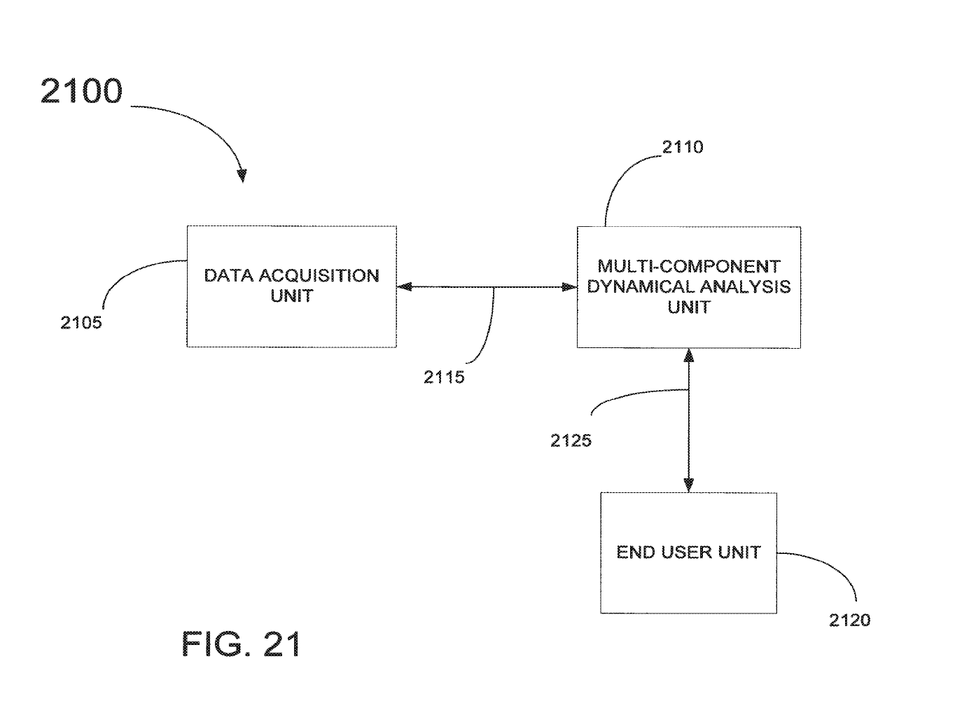

FIG. 21 illustrates an example system 2100 for monitoring dynamical behavior of a multi-component system in accordance with exemplary embodiments of the present methods. For example, the system 2100 is configured to implement the methods or corresponding algorithms described above.

The System 2100 includes a data acquisition unit or device 2105 for a multi-component system 2105, a multi-component dynamical analysis unit or device 2110, and an optional end user unit or device 2120. Data acquisition device 2105 is coupled to multi-component dynamical analysis device 2110 by a communications medium (e.g., a wired or wireless communications line) 2115. Similarly, the multi-component dynamical analysis device 2110 is coupled to the end user device 2120 by a communications medium (e.g., a wired or wireless communications line) 2125.

Data acquisition device 2105 monitors incoming signals from one or more sensors or electrodes of the multi-component system. The multi-component dynamical analysis device 2110 acquires the incoming signals from data acquisition device 2105 over communications medium 2115. The multi-component dynamical analysis device 2110 continuously calculates or evaluates various signal measures such as signal energy, approximate entropy, dynamical phase, maximum short-term Lyapunov exponent (STLₘₐₓ), among other measures, for each electrode site through analysis of sequential overlapping or non-overlapping time windows of variable lengths.

By implementing the techniques for monitoring dynamical behavior described herein, multi-component dynamical analysis device 2110 can detect temporal, spatial, or spatiotemporal patterns and trends of each signal, signal properties, and interactions described above. Multi-component dynamical analysis device 2110 can also detect states, state transitions, and self-organizing patterns. Furthermore, the multi-component dynamical analysis device 2110 can detect evidence for deterministic (linear or nonlinear) and/or stochastic processes and can be used to predict short and long term trends and state transitions.

In an embodiment, the multi-component dynamical analysis device 2110 may include an analysis workstation or a handheld device capable of real time visualization of the acquired signals, the dynamic measure calculations, and the statistical analysis with real time event prediction and event detection. The multi-component dynamical analysis device 2110 may include a computational module which may be a computer-readable medium containing a program adapted to cause a data processing system to execute the above-noted method steps. In this regard, the computer-readable medium may be a computer-readable medium, such as solid-state memory, magnetic memory such as a magnetic disk, optical memory such as an optical disk, or a computer-readable transmission medium.

Analysis results can be displayed graphically or numerically on any desirable medium (such as a display screen or a printer, for example) for purposes of long term monitoring, diagnostics, and prediction. Optionally, the multi-component dynamical analysis device 2110 communicates analysis results to end user device 2120 over communications medium 2125. In this way, real time event prediction and event detection, in addition to event intervention, can be communicated to the end user device 2120.

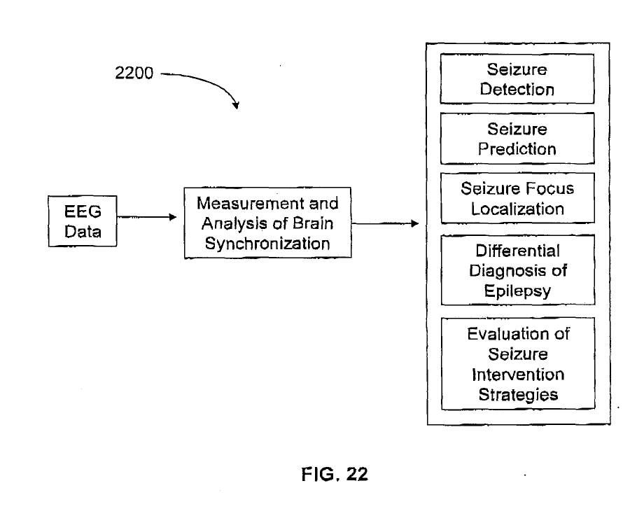

Applications of the multi-component dynamical analysis methods described above include investigating EEG's of patients with epilepsy for seizure prediction, seizure detection, seizure focus localization, differential diagnosis of epilepsy, and evaluation of seizure intervention strategies, among others, as shown in FIG. 22.

The system 2100 may also be configured as an on-line system that incorporates the various features and applications described above. The system 2100 may be used in any number of clinical or non-clinical applications, including diagnostic applications, as well as applications relating to patient treatment. For example, the system 2100 may be used to collect and process EEG signals for subsequent clinical interpretation (e.g., to analyze and determine seizure propagation patterns). The system 2100 may also be used to alert hospital or clinic staff members of an impending seizure, via a local or telemetry link, so that staff members have adequate time to prevent patient injury or provide timely medical intervention to prevent the seizure itself; to observe the seizure, or to prepare for and administer other procedures that must be accomplished during the seizure, such as the administration of radiolabelled ligands or other substances required to obtain data and/or images for pre-surgical diagnostic purposes.

In addition to surgical excision of the epileptogenic focus, current methods for controlling epileptic seizures include pharmacological (i.e., antiepileptic drug) therapy. The currently accepted pharmacological approach is to prescribe fixed doses of one or more antiepileptic drugs to be taken chronically at fixed time intervals. The objective is to achieve a steady-state concentration in the brain that is high enough to provide optimal seizure control, but low enough to reduce the risk of side-effects.

Another embodiment of the system 2100 may include an indwelling or implantable device in the patient, such as a real-time digital signal processing chip (not shown), that contains, among other things, above described algorithms to provide seizure warning and prediction. For example, the system 2100 may be configure to alert the patient of any potentially impending seizure and/or to trigger the release of a compound, such as a small dose of an anticonvulsant drug, into the blood stream of the patient from a stimulator (not shown) which contains or is connected to an indwelling reservoir. The objective, of course, is to release a small quantity of anticonvulsant drug during the preictal transition stage to abort the impending seizure.

Another embodiment of the system 2100 may be configured to deliver, in addition to anticonvulsant drug therapy, electric or magnetic stimulation, for example, through a vagal nerve stimulator. This exemplary embodiment of the system 2100 may then delivers an electrical impulse to the vagus nerve in the neck of specified duration and intensity, but only during the preictal transition state. To accomplish this objective, the system 2100 is configured to detect the preictal transition state based on dynamical analysis of ongoing brain electrical activity, as described in detail above. When a preictal state is detected, the indwelling vagal nerve stimulator is triggered and an electrical pulse is delivered to the vagus nerve in the neck. It will be readily apparent, however, to those skilled in the art that devices other than vagal nerve stimulators, for example, deep brain stimulators, may be used in conjunction with the present invention to create brain pacemakers for epileptic patients.

All of the methods and apparatus disclosed and claimed herein can be made and executed without undue experimentation in light of the present disclosure. While the compositions and methods of this invention have been described in terms of preferred embodiments, it will be apparent to those of skill in the art that variations may be applied to the methods and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for analyzing a multi-component system, the method comprising:
acquiring a plurality of signals, each signal associated with a different spatial location of a portion of the multi-component system;
generating a plurality of dynamic profiles for each of the plurality of signals, each of the plurality of dynamic profiles reflecting dynamic characteristics of the corresponding signal in accordance with each one of a plurality of dynamic measures;
selecting a plurality of pairs of dynamic profiles from the acquired plurality of dynamic profiles based on a predetermined level of synchronization;
generating a statistical measure for each of the selected plurality of pairs of dynamic profiles;
characterizing state dynamics of the multi-component system as a function of at least one of the generated statistical measures; and
generating a signal indicative of the characterized state dynamics of the multi-component system.

2. The method of claim 1, wherein the generated signal is used to at least one of monitor dynamic transitions of the multi-component system, identify spatial locations of interest in the multi-component system, differentiate the state dynamics of the multi-component system from other dynamics of similar systems, evaluate and determine a treatment efficacy for the multi-component system, and identify a susceptibility of the multi-component system to a predetermined condition.

3. The method of claim 2, wherein the spatial locations of interest are pathological spatial locations of a brain of a patient.

4. The method of claim 2, wherein the state dynamics identify pathologies.

5. The method of claim 2, wherein the treatment regimen is dependent on the pathology of a patient.

6. The method of claim 2, wherein the predetermined condition is a pathology of a patient.

7. The method of claim 1, further comprising comparing each of the generated statistical measures to a predetermined threshold value and characterizing an evolution of the state dynamics of the multi-component system based on the comparison.

8. The method of claim 7, wherein the generated statistical measures are tracked to monitor the evolution of the state dynamics.

9. The method of claim 3, wherein the multi-component system is an epileptic brain and the signal is a seizure warning when the generated statistical measures exceed at least one of the predetermined threshold values.

10. The method of claim 1, wherein the order of synchronization of the plurality of generated statistical measures is determined to characterize the state dynamics of the multi-component system.

11. The method of claim 8, wherein the tracked statistical measures are a Lyapunov exponent measure, a phase measure and an energy measure, each of which is derivable from a mathematical representation of each of the plurality of signals.

12. The method of claim 11, wherein the tracking comprises tracking based on the Lyapunov exponent measure of each of the plurality of signals.

13. The method of claim 11, wherein the tracking comprises tracking based on the phase measure of the signals.

14. The method of claim 11, wherein the tracking comprises tracking based on the energy measure of the signals.

15. The method of claim 11, wherein the generating of the indicative signal is dependent on a predetermined sequential synchronization of the statistical measures.

16. The method of claim 15, wherein the predetermined sequential synchronization of the statistical measures involves a synchronization of the Lyapunov exponent measure followed by a synchronization of the phase measure which in turn is followed by a synchronization of the energy measure.

17. The method of claim 1, further comprising determining an amount of synchronization by evaluating a ratio of the number of the selected plurality of pairs of signals to the total number of the plurality of pairs of signals.

18. The method of claim 2, wherein the monitoring of the dynamic transitions of the multi-component system comprises determining whether the generated signal exceeds a predetermined threshold.

19. The method of claim 2, wherein the identifying of the spatial locations of interest in the multi-component system comprises using the frequency of the resetting and/or synchronization of selected pairs of dynamic profiles.

20. The method of claim 2, wherein the differentiation of the state dynamics of the multi-component system from other dynamics of similar systems comprises comparing the generated signal to a predetermined threshold.

21. The method of claim 2, wherein evaluating and determining treatment efficacy for the multi-component system is dependent on the level of the generated signal.

22. The method of claim 2, wherein identifying a susceptibility of the multi-component system to a predetermined condition is dependent on the level of the generated signal.

23. The method of claim 1, wherein selecting a plurality of pairs of dynamic profiles does not involve any training in the computation of the dynamic measures.

24. The method of claim 1, wherein characterizing state dynamics does not involve any training in the computation of the statistical measures.

25. The method of claim 1, wherein the plurality of dynamic measures are patient independent.

26. The method of claim 1, wherein selecting a plurality of pairs of dynamic profiles from the acquired plurality of dynamic profiles is not initiated based on an evaluation of a predetermined reference value of the dynamic measures.

27. The method of claim 1, further comprising determining a level of resetting of the multi-component system by evaluating a difference of averaged synchronization values of the selected plurality of pairs of dynamic profiles for a period of time before and after the generation of the indicative signal.

28. The method of claim 1, further comprising determining a rate of resetting by evaluating a ratio of a time taken by the selected plurality of pairs of dynamic profiles to no longer satisfy the predetermined level of synchronization after the generation of the indicative signal over a time during which the selected plurality of pairs of dynamic profiles satisfy the predetermined level of synchronization

29. The method of claim 1, further comprising determining a frequency of Resetting by determining the number of generations of the indicative signal over a period of time.

30. The method of claim 3, further comprising determining a focus of a seizure by identifying the spatial locations corresponding to the pair of dynamic profiles having the largest amount of synchronization for a length of time.

31. The method of claim 1, wherein acquiring a plurality of signals further comprises processing and filtering the plurality of signals.

32. A computer-readable medium containing a computer program adapted to cause a computer to execute a method for analyzing a multi-component system, comprising:
acquiring a plurality of signals, each signal associated with a different spatial location of a portion of the multi-component system;
generating a plurality of dynamic profiles for each of the plurality of signals, each of the plurality of dynamic profiles reflecting dynamic characteristics of the corresponding signal in accordance with each one of a plurality of dynamic measures;
selecting a plurality of pairs of dynamic profiles from the acquired plurality of dynamic profiles based on a predetermined level of synchronization;
generating a statistical measure for each of the selected plurality of pairs of dynamic profiles;
characterizing state dynamics of the multi-component system as a function of at least one of the generated statistical measures; and
generating a signal indicative of the characterized state dynamics of the multi-component system.

33. A system for analyzing a multi-component system, comprising:
a data acquisition unit acquiring a plurality of signals, each signal associated with a different spatial location of a portion of the multi-component system;
an analysis unit for generating a plurality of dynamic profiles for each of the plurality of signals, each of the plurality of dynamic profiles reflecting dynamic characteristics of the corresponding signal in accordance with each one of a plurality of dynamic measures, selecting a plurality of pairs of dynamic profiles from the acquired plurality of dynamic profiles based on a predetermined level of synchronization, generating a statistical measure for each of the selected plurality of pairs of dynamic profiles, characterizing state dynamics of the multi-component system as a function of at least one of the generated statistical measures, and generating a signal indicative of the characterized state dynamics of the multi-component system; and
an end user unit capable for displaying analytical results.

34. The system of claim 33, wherein the data acquisition unit is a unit implantable in a brain of a patient.

35. The system of claim 33, wherein the data acquisition unit further comprises electrodes, each of the electrodes having a location corresponding to one of the different spatial locations of the portion of the multi-component system.

36. The system of claim 33, wherein the end user unit is configured to interfere with a proceeding of the brain towards a seizure.

37. The system of claim 34, wherein the analysis unit is a unit implantable in the brain of the patient.
